(19) United States
(12) Patent Application Publication
(10) Pub. No.: US 2025/0075170 A1
(43) Pub. Date: Mar. 6, 2025

(54) TARGETED MUTAGENESIS IN SPIRULINA

(71) Applicant: Lumen Bioscience, Inc., Seattle, WA (US)

(72) Inventors: Ryo TAKEUCHI, Seattle, WA (US); James ROBERTS, Seattle, WA (US)

(21) Appl. No.: 18/767,011

(22) Filed: Jul. 9, 2024

Related U.S. Application Data
(63) Continuation of application No. 16/570,520, filed on Sep. 13, 2019, now Pat. No. 12,065,638.
Application No. 16/570,520 is a continuation of application No. 16/122,250, filed on Sep. 5, 2018, now Pat. No. 10,415,013.
Application No. 16/122,250 is a continuation of application No. 15/510,028, filed on Mar. 9, 2017, now Pat. No. 10,131,870.
(60) Provisional application No. 62/047,811, filed on Sep. 9, 2014.

(51) Int. Cl. C12N 1/20; C07K 14/195; C12N 13/00; C12N 15/74; C12N 15/90

(52) U.S. Cl. CPC C12N 1/20; C07K 14/195; C12N 13/00; C12N 15/74; C12N 15/902

(57) ABSTRACT

This disclosure describes techniques for creating stable, targeted mutations in Spirulina (Athrospiria) and Spirulina having stable, targeted mutations.

### PRIORITY CLAIM

This application is a continuation of U.S. application Ser. No. 16/570,520, filed Sep. 13, 2019; which is a continuation of U.S. application Ser. No. 16/122,250, filed Sep. 5, 2018 (now U.S. Pat. No. 10,415,013), which is a continuation of U.S. application Ser. No. 15/510,028, filed Mar. 9, 2017 (now U.S. Pat. No. 10,131,870), which is a national stage application of international patent application No. PCT/US15/49214, entitled “Targeted Mutagenesis In Spirulina,” and filed Sep. 9, 2015, which claims priority to U.S. Provisional Patent Application No. 62/047,811, entitled “Targeted Mutagenesis in Spirulina,” and filed Sep. 9, 2014, the contents of each of which are incorporated in their entirety.

### SEQUENCE LISTING

The contents of the electronic sequence listing (LUBI_006_06US_SeqList_ST26.xml; Size: 77,964 bytes; and Date of Creation: Jul. 5, 2024) are herein incorporated by reference in its entirety.

### BACKGROUND

Cyanobacteria, also called blue-green algae, are photosynthetic organisms that use chlorophyll A and water to reduce carbon dioxide and generate energy-containing compounds. Spirulina are free-floating, filamentous cyanobacteria that include the species Arthrospira platensis and Arthrospira maxima. These two species were formerly classified in the genius Spirulina, but are now classified in the genus Arthrospira. However, the term “Spirulina” remains in use.

Cyanobacteria are generally amenable to genetic manipulation. However, genetic engineering tools for Spirulina are limited. Many techniques for genetic manipulation are based on introducing exogenous (foreign) genetic material into a bacterial cell. Cells must be in a state of “competence” to take up genetic material from the surrounding environment. Some types of bacteria are able to naturally take up genetic material. These types of bacteria are referred to as having “natural competence.” More commonly, “artificial competence” is induced by making a cell temporarily permeable to genetic material. Techniques for introducing artificial competence by increasing the permeability of an outer cell membrane include incubation in chemical solutions, heat shock, and electroporation which subjects a cell to an electric field. A cell in a state of competence that uptakes exogenous genetic material and incorporates the new genetic material into its genome is said to be have undergone “transformation.”

Spirulina has been long recognized as difficult to transform by random integration of DNA into a Spirulina chromosome, and applicant is not aware of any reports claiming modification of the Spirulina genome by targeted introduction of DNA into specific, predetermined chromosome locations. Attempts using electroporation to introduce a gene for chloramphenicol resistance have resulted in chloramphenicol resistance under certain electroporation conditions, but the transformation was not stable (i.e. the chloramphenicol resistance could not be sustained). Subsequent attempts to transform Spirulina with a gene for chloramphenicol resistance coupled to a strong promoter by using electroporation achieved cells that grew in the presence of chloramphenicol for 12 months, but the method only allowed the gene for chloramphenicol resistance to be located at random (un-targeted) locations in a Spirulina chromosome, and even this random integration of exogenous DNA was not conclusively demonstrated. Recently, random mutagenesis has been achieved in S. platensis (A. platensis) using atmospheric and room-temperature plasma (ARTP). However, random mutagenesis does not transform the Spirulina cells through introducing exogenous genetic material, rather mutations are introduced at random sites in the genome. A lack of understanding of how to stably introduce foreign DNA to predetermined chromosome locations into cyanobacteria, Spirulina in particular, is recognized as a challenge to working with cyanobacteria as compared to other organisms such as E. coli or yeast. Although there was some level of success with random mutagenesis, this research also highlighted the continued lack of an effective system for mutation of S. plantensis by introduction and expression an exogenous gene. Moreover, none of the techniques described above have attempted to introduce targeted mutations to specific, pre-determined regions of the Spirulina genome.

A need still exists for a technique to efficiently create stable transformants in Spirulina. Moreover, there is also a need for techniques that allow for targeted introduction of mutations in the Spirulina genome.

### BRIEF SUMMARY

This disclosure describes techniques for introducing stable, targeted mutations to the genome of Spirulina. This disclosure also describes Spirulina modified to contain stable, targeted mutations. Additionally, this disclosure described techniques for using Spirulina that have been modified to include one or more stable, targeted mutation to manufacture products of interest.

This disclosure describes methods of creating targeted mutations in Spirulina by contacting the Spirulina with an osmotic stabilizer, contacting the Spirulina with a vector having homology arms, and inducing artificial competence in the Spirulina. In an embodiment, the Spirulina may be Arthrospira platensis NIES-39 or Arthrospira sp. PCC 8005. The osmotic stabilizer may be, but is not limited to, polyethylene glycol (PEG), ethylene glycol, glycerol, glucose, sucrose, or any combination thereof. The vector may be a DNA vector, a linear vector, a circular vector, a single stranded polynucleotide, or a double stranded polynucleotide. In an embodiment the vector may be, for example, a pApl-pilA/aadA plasmid.

In an embodiment the homology arms may be the same length. In an embodiment the homology arms may be different lengths. One or both of the homology arms may be at least about 500 bp, at least about 1000 bp, at least about 1500 bp, or at least about 2000 bp.

In an embodiment the artificial competence may be induced by any known technique for introducing artificial competence in prokaryotic cells including incubation in a solution containing divalent cations, electroporation, and ultrasound.

In an embodiment, the method may also include contacting the Spirulina with a pH balancer. The Spirulina may be contacted by the pH balancer prior to or after contacting the Spirulina with the vector. In an embodiment a pH of the Spirulina after contacting with the pH balancer is about 7.0 to about 8.0.

This disclosure describes Spirulina comprising at least one stable, targeted mutation. In an embodiment the Spirulina is Arthrospira platensis, Arthrospira platensis NIES-39, or Arthrospira maxima. In an embodiment, the stable targeted mutation is inherited for at least 5 generations, at least 10 generations, at least 20 generations, at least 30 generations, at least 40 generations, or at least 50 generations. The mutation may be any type of alteration to the genome including a deletion or disruption of at least a portion of a gene, an insertion of an additional copy of an endogenous gene, or addition of an exogenous gene. In an embodiment, the targeted mutation may include addition of an exogenous protein domain including post-translational modification sites, protein-stabilizing domains, cellular localization signals, and protein-protein interaction domains. In an embodiment, the targeted mutation comprises addition of a nucleic acid sequence that is not translated into a protein including, but not limited to, a non-coding RNA molecule, a gene regulatory element, a promoter, a regulatory protein binding site, a RNA binding site, a ribosome binding site, a transcriptional terminator, or a RNA-stabilizing element.

This disclosure also describes a Spirulina cell lacking at least one protein as a result of introducing a modification at the loci of the protein by transformation of the Spirulina cell with at least one DNA construct comprising a sequence homologous with at least a portion of the loci and the modification and integration of the DNA construct at the loci, the Spirulina cell being otherwise capable of functioning in its native manner.

### BRIEF DESCRIPTION OF THE FIGURES

FIG. 1 shows left and right homology arms designed to target the pilA gene locus (NIES39_C03030) in A. platensis strain NIES-39.

FIG. 2 shows a pApl-pilA/aadA plasmid used for targeted mutagenesis in A. platensis strain NIES-39. The plasmid of FIG. 2 has nucleotide sequence SEQ ID NO: 1.

FIG. 3A shows a schematic of homologous recombination at the pilA gene locus (NIES39_C03030) in A. platensis strain NIES-39 with the plasmid of FIG. 2 containing the aminoglycoside adenyltransferase gene (aadA). Five PCR primer sites labeled A, B, C, D, E, F, and G are also shown.

FIG. 3B shows the results of PCR amplification with primers C/E, A/D, and B/C for both wild-type A. platensis and A. platensis following transformation with the plasmid of FIG. 2.

FIG. 3C shows the results of PCR amplification with primers F/G of the pilA gene sequence in wild-type A. platensis and lack of amplification in A. platensis following transformation with the vector containing aadA.

FIG. 4 shows a pApl-pilA8005/aadA plasmid used for targeted mutagenesis in Arthrospira sp. PCC 8005. The plasmid of FIG. 4 has nucleotide sequence SEQ ID NO: 2.

FIG. 5A shows a schematic of homologous recombination at the pilA gene locus in Arthrospira sp. PCC 8005 with the plasmid of FIG. 4 containing the aminoglycoside adenyltransferase gene (aadA). Three PCR primer sites labeled H, I, and J are also shown.

FIG. 5B shows the results of PCR amplification with primers H/I and H/J for both wild-type Arthrospira sp. PCC 8005 and Arthrospira sp. PCC 8005 following transformation with the plasmid of FIG. 4.

FIG. 6 shows a pApl-NS1/Prs-crtW-crtZ plasmid used to attempt to introduce crtW and crtZ genes by targeted mutagenesis in A. platensis strain NIES-39 to enable A. platensis to synthesize astaxanthin. The plasmid of FIG. 6 has nucleotide sequence SEQ ID NO: 3.

FIG. 7 shows a pApl-NS1/aadA-cpcBA plasmid used to introduce cpcA and cpcB genes by targeted mutagenesis in A. platensis strain NIES-39 to add additional copies of these two genes to Athrospira. This results in an Athrospira strain that overproduces C-phycocyanin. The plasmid of FIG. 7 has the nucleotide sequence SEQ ID NO: 4.

FIGS. 8A-8C shows improvements in C-phycocyanin production in A. platensis strain NIES-39 transformed with the plasmid of FIG. 7 as compared to wild-type A. platensis. FIG. 8D shows that the transformed A. platensis has a growth rate similar to wild-type A. platensis.

FIG. 9A shows a deeper blue color in a protein extraction from A. platensis strain NIES-39 transformed to overproduce C-phycocyanin and a lighter blue protein extraction from wild-type A. platensis strain NIES-39.

FIG. 9B is a graph showing absorption differences between the protein extractions from the transformed A. platensis strain NIES-39 and wild-type A. platensis strain NIES-39. Transformed A. platensis strain NIES-39 has greater absorption at 620 nm than the wild-type strain.

### DETAILED DESCRIPTION

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, illustrative methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

As used herein, the terms “having,” “has,” “contain,” “including,” “includes,” “include,” and “have” have the same open-ended meaning as “comprising,” “comprises,” and “comprise” provided above.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

By “about” is meant a quantity, level, value, number, frequency, percentage, dimension, size, amount, weight or length that varies by as much as 30, 25, 20, 15, 10, 9, 8, 7, 6, 5, 4, 3, 2, or 1% to a reference quantity, level, value, number, frequency, percentage, dimension, size, amount, weight, or length.

The present description uses numerical ranges to quantify certain parameters relating to the invention. It should be understood that when numerical ranges are provided, such ranges are to be construed as providing literal support for claim limitations that only recite the lower value of the range as well as claim limitations that only recite the upper value of the range. For example, a disclosed numerical range of 10 to 100 provides literal support for a claim reciting “greater than 10” (with no upper bounds) and a claim reciting “less than 100” (with no lower bounds) and provided literal support for and includes the end points of 10 and 100.

The present description uses specific numerical values to quantify certain parameters relating to the invention, where the specific numerical values are not expressly part of a numerical range. It should be understood that each specific numerical value provided herein is to be construed as providing literal support for a broad, intermediate, and narrow range. The broad range associated with each specific numerical value is the numerical value plus and minus 60 percent of the numerical value, rounded to two significant digits. The intermediate range associated with each specific numerical value is the numerical value plus and minus 30 percent of the numerical value, rounded to two significant digits. The narrow range associated with each specific numerical value is the numerical value plus and minus 15 percent of the numerical value, rounded to two significant digits. These broad, intermediate, and narrow numerical ranges should be applied not only to the specific values, but should also be applied to differences between these specific values.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences) whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The recitation “polynucleotide” or “nucleic acid” as used herein designates mRNA, RNA, CRNA, rRNA, cDNA, or DNA. The term typically refers to polymeric form of nucleotides of at least 10 bases in length, either ribonucleotides or deoxynucleotides or a modified form of either type of nucleotide. The term includes single and double stranded forms of DNA and RNA.

As used herein, the term “DNA” includes a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

With regard to polynucleotides, the term “exogenous” refers to a polynucleotide sequence that does not naturally occur in a wild-type cell or organism, but is typically introduced into the cell by molecular biological techniques. Examples of exogenous polynucleotides include vectors, plasmids, and/or man-made nucleic acid constructs encoding a desired protein. With regard to polynucleotides, the term “endogenous” or “native” refers to naturally occurring polynucleotide sequences that may be found in a given wild-type cell or organism. A vector, plasmid, or other man-made construct that includes an endogenous polynucleotide sequence combined with polynucleotide sequences of the unmodified vector etc. is, as a whole, an exogenous polynucleotide and may also be referred to as an exogenous polynucleotide including an endogenous polynucleotide sequence. Also, a particular polynucleotide sequence that is isolated from a first organism and transferred to second organism by molecular biological techniques is typically considered an “exogenous” polynucleotide with respect to the second organism.

Polynucleotides may comprise a native sequence (e.g., an endogenous sequence that encodes protein described herein) or may comprise a variant or fragment, or a biological functional equivalent of such a sequence. Polynucleotide variants may contain one or more substitutions, additions, deletions and/or insertions, as further described herein, preferably such that the enzymatic activity of the encoded polypeptide is not substantially diminished relative to the unmodified or reference polypeptide. The effect on the enzymatic activity of the encoded polypeptide may generally be assessed as described herein and known in the art.

As will be understood by those skilled in the art, the polynucleotide sequences of this disclosure can include genomic sequences, extra-genomic, and plasmid-encoded sequences and smaller engineered gene segments that express, or may be adapted to express, proteins, polypeptides, peptides and the like. Such segments may be naturally isolated, or modified synthetically by the hand of man.

Polynucleotides may be single-stranded (coding or antisense) or double-stranded, and may be DNA (genomic, cDNA, or synthetic) or RNA molecules. Additional coding or non-coding sequences may, but need not, be present within a polynucleotide of the present invention, and a polynucleotide may, but need not, be linked to other molecules and/or support materials.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

The percent identity of two sequences, whether nucleic acid or amino acid sequences, is the number of exact matches between two aligned sequences divided by the length of the shorter sequences and multiplied by 100. An approximate alignment for nucleic acid sequences is provided by the Smith-Waterman algorithm. The Smith-Waterman algorithm can be applied to amino acid sequences by using a known scoring matrix (e.g., the scoring matrix developed by Dayhoff) and normalized by any well-known technique such as the Gribskov method. One implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). Other suitable programs for calculating the percent identity or similarity between sequences are generally known in the art, for example, the MPSRCH package of programs copyrighted by the University of Edinburgh, developed by John F. Collins and Shane S. Sturrok, and distributed by IntelliGenetics, Inc. (Mountain View, Calif.), and BLAST, used with default parameters. Details of these programs can be found at the following internet address: blast.ncbi.nlm.nih.gov/Blast.cgi.

“Polypeptide,” “polypeptide fragment,” “peptide,” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions. Exemplary nucleotide sequences that encode the proteins and enzymes of the application encompass full-length reference polynucleotides, as well as portions of the full-length or substantially full-length nucleotide sequences of these genes or their transcripts or DNA copies of these transcripts. Portions of a nucleotide sequence may encode polypeptide portions or segments that retain the biological activity of the reference polypeptide.

“Transformation” refers to the stable, heritable alteration in a cell resulting from the uptake and incorporation of exogenous nucleotides into the host-cell genome; also, the transfer of an exogenous gene from one organism into the genome of another organism. Exogenous nucleotides may include gene foreign to the target organism or addition of a nucleotide sequence present in the wild-type organism.

“Targeted mutation” means a change in the DNA sequence of the genome at a pre-determined (specified) genome location. In some cases a targeted mutation will involve the introduction of a pre-determined (specified) DNA sequence alteration at the pre-determined genome location. In other cases a targeted mutation will involve the introduction of a random DNA sequence alteration at the pre-determined genome location.

“Stable” when describing the results of a genetic modification caused by transformation refers to a genetic modification that is maintained in at least a portion of a population of cells for ten or more generations or for a length of time equal or greater to ten times the average generation time for the modified organism.

“Competent” refers to the ability of a cell to take up extracellular nucleotides from the surrounding environment. A cell may be “naturally competent” or “artificially competent.” Naturally competent cells are able to take up nucleotides from their surrounding environment under natural conditions. Artificially competent cells are made passively permeable to extracellular nucleotides by exposing the cell to conditions that do not normally occur naturally including incubation in a solution of divalent cations, heat shock, electroporation, and ultrasound.

The terms “wild-type” and “naturally occurring” are used interchangeably to refer to an organism, gene, or gene product that has the characteristics of that organism, gene or gene product (e.g., a polypeptide) when isolated from a naturally occurring source. A wild-type organism, gene, or gene product is that which is most frequently observed in a population and is thus arbitrarily designed the “normal” or “wild-type” form.

As used herein “Spirulina” is synonymous with “Arthrospira.” The genus Arthrospria includes 57 species of which 22 are currently taxonomically accepted. Thus, reference to “Spirulina” or “Arthrospira” without further designation includes reference to any of the following species: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor.

All literature and similar materials cited in this application, including patents, patent applications, articles, books, treatises, and Internet web pages are expressly incorporated by reference in their entirety for any purpose. When definitions of terms in incorporated references appear to differ from the definitions provided in this application, the definition provided in this application shall control.

### Targeted Mutations in Spirulina

In an aspect of the invention, a vector having homology arms is taken up by a Spirulina cell in a state of competence and subsequently integrated into one or more chromosomes of the cell. Homologous recombination guided by the sequences of the homology arms changes the genome of the cell due to differences between the original nucleic acid sequence of the genome and the nucleic acid sequence of the vector region between the homology arms.

The present disclosure describes the first technique known to the inventors for inducing competence in Spirulina. Spirulina is not naturally competent and the techniques disclosed herein achieve a transformation where previous techniques for transforming Spirulina have failed. The high levels of endonucleases present in Spirulina have been previously thought to make transformation impossible. Electroporation has been limited to creating competent cells for only brief periods of time due to the tendency of Spirulina cells to lyse when subjected to electroporation. However, transformation by electroporation in the presence of an appropriate osmotic stabilizer achieves transformation that was previously not possible by other techniques.

Prior to transformation, Spirulina may be cultured and washed with an osmotic stabilizer. Spirulina may be cultured in any suitable media for growth of cyanobacteria such as SOT medium. SOT medium includes NaHCO₃ 1.68 g, K₂HPO₄ 50 mg, NaNO₃ 250 mg, K₂SO₄ 100 mg, NaCl 100 mg, MgSO₄·7H₂O, 20 mg, CaCl₂·2H₂O 4 mg, FeSO₄·7H₂O 1 mg, Na₂EDTA·2H₂O 8 mg, A₅ solution 0.1 mL, and distilled water 99.9 mL. A₅ solution includes H₃BO₃ 286 mg, MnSO₄·5H₂O) 217 mg, ZnSO₄·7H₂O 22.2 mg, CuSO₄·5H₂O 7.9 mg, Na₂MoO₄·2H₂O 2.1 mg, and distilled water 100 mL. Cultivation may occur with shaking (e.g., 100-300 rpm) at a temperature higher than room temperature (e.g. 25-37° C.) and under continuous illumination (e.g. 20-2,000, 50-500, or 100-200 μmol photon m⁻² s⁻¹). The growing cells may be harvested when the optical density at 750 nm reaches a predetermined threshold (e.g., OD₇₅₀ of 0.3-2.0, 0.5-1.0, or 0.6-0.8). A volume of the harvested cells may be concentrated by centrifugation then resuspended in a solution of pH balancer and salt. The pH balancer may be any suitable buffer that maintains viability of Spirulina while keeping pH of the media between 6 and 9 pH, between 6.5 and 8.5 pH, or between 7 and 8 pH. Suitable pH balancers include HEPES, HEPES-NaOH, sodium or potassium phosphate buffer, and TES. The salt solution may be NaCl at a concentration of between 50 mM and 500 mM, between 100 mM and 400 mM, or between 200 mM and 300 mM. In an embodiment between 1-50 mL of 1-100 mM PH balance may be used to neutralize the pH.

Cells collected by centrifugation may be washed with an osmotic stabilizer and optionally a salt solution (e.g. 1-50 ml of 0.1-100 mM NaCl). Any amount of the culture may be concentrated by centrifugation. In an embodiment between 5-500 ml of the culture may be centrifuged. The osmotic stabilizer may be any type of osmotic balancer that stabilizes cell integrity of Spriulina during electroporation. In an embodiment, the osmotic stabilizer may be a sugar (e.g. w/v 0.1-25%) such as glucose or sucrose. In an embodiment the osmotic stabilizer may be a simple polyol (e.g. v/v 1-25%) including glycerine, glycerin, or glycerol. In an embodiment the osmotic stabilizer may be a polyether including (e.g. w/v 0.1-20%) polyethylene glycol (PEG), poly(oxyethylene), or poly(ethylene oxide) (PEO). The PEG or PEO may have any molecular weight from 200 to 10,000, from 1000 to 6000, or from 2000 to 4000. In an embodiment the pH balancer or buffer may be used instead of or in addition to the osmotic stabilizer.

The present disclosure also describes creation of targeted mutations in Spirulina through homologous recombination by introduction of a vector to competent cells. Artificial competency may be created by the electroporation technique described above or by any other known or future technique for creating competency in Spirulina. Known techniques for introducing artificial competence in Spirulina include electroporation (with or without an osmotic stabilizer and with or without a pH balancer), incubation in a solution containing divalent cations, and ultrasound. The Spirulina cells are contacted by a vector when artificial competency is induced. For example, a vector may be mixed with a solution of Spirulina cells prior to electroporation.

Electroporation may be performed in a 0.1-, 0.2- or 0.4-cm electroporation cuvette at between 0.6 and 10 kV/cm, between 2.5 and 6.5 kV/cm, or between 4.0 and 5.0 kV/cm; between 1 and 100 μF, between 30 and 70 μF, or between 45 and 55 μF; and between 10 and 500 mΩ, between 50 and 250 mΩ, or between 90 and 110 mΩ. In an embodiment electroporation may be performed at 4.5 kV/cm, 50 μF, and 100 mΩ.

Following electroporation the cells may be grown in the presence of one or more antibiotics selected based on resistance conferred through successful transformation with the plasmid. Post-electroporation culturing may be performed at reduced illumination levels (e.g. 5-500, 10-100, or 30-60 μmol photon m⁻² s⁻¹). The culturing may also be performed with shaking (e.g. 100-300 rpm). The level of antibiotics in the media may be between 5 and 100 μg/mL. Post-electroporation culturing may be continued for 1-5 days or longer. Successful transformants identified by antibiotic resistance may be selected over a time course of 1 week to 1 month on plates or in 5-100 ml of SOT medium supplemented with 0.1-2.0 μg of appropriate antibiotics.

By “vector” is meant a polynucleotide molecule, preferably a DNA molecule derived, for example, from a plasmid, bacteriophage, yeast, or virus, into which a polynucleotide can be inserted or cloned. A vector may contain one or more synthetic nucleotides or nucleic acid analogues. A vector may contain one or more unique restriction sites. The vector can be one which, when introduced into the host cell, is integrated into the genome and replicated together with the chromosome(s) into which it has been integrated. Such a vector may comprise one or more specific sequences that allow recombination into a particular, desired site of the host chromosome. These specific sequences may be homologous to sequences present in the wild-type genome. A vector system can comprise a single vector or plasmid, two or more vectors or plasmids, some of which increase the efficiency of targeted mutagenesis, or a transposition. The choice of the vector will typically depend on the compatibility of the vector with the host cell into which the vector is to be introduced. The vector can include a reporter gene, such as a green fluorescent protein (GFP), which can be either fused in frame to one or more of the encoded polypeptides, or expressed separately. The vector can also include a positive selection marker such as an antibiotic resistance gene that can be used for selection of suitable transformants. The vector can also include a negative selection marker such as the type II thioesterase (tesA) gene or the Bacillus subtilis structural gene (sacB). Use of a reporter or marker allows for identification of those cells that have been successfully transformed with the vector.

In an embodiment, the vector includes one or two homology arms that are homologous to DNA sequences of the Spirulina genome which are adjacent to the targeted locus. The sequence of the homology arms may be identical or similar to the regions of the Spirulina genome to which the homology arms are complementary. “Homology” or “homologous” as used herein includes both homologous, identical sequences and homologous, non-identical sequences. Homologous non-identical sequences refer to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. As used herein, the degree of homology between the two homologous, non-identical sequences is sufficient to allow homologous recombination there between, utilizing normal cellular mechanisms. Two homologous non-identical sequences can be any length and their degree of non-homology can be as small as a single nucleotide (e.g., for a genomic point mutation introduced targeted homologous recombination) or as large as 10 or more kilobases (e.g., for insertion of a gene at a predetermined locus in a chromosome). Two polynucleotides comprising homologous non-identical sequences need not be the same length. For example, an exogenous polynucleotide (i.e., vector polynucleotide) of between 20 and 10,000 nucleotides or nucleotide pairs can be used.

The characterization of two sequences as homologous, identical sequences or homologous, non-identical sequences may be determined by comparing the percent identity between the two sequences (polynucleotide or amino acid). Homologous, identical sequences have 100% sequence identity. Homologous, non-identical sequences may have sequence identity greater than 80%, greater than 85%, greater than 90%, greater than 91%, greater than 92%, greater than 93%, greater than 94%, greater than 95%, greater than 96%, greater than 97%, greater than 98%, or greater than 99%.

The homology arms may be any length that allows for site-specific homologous recombination. A homology arm may be any length between about 2000 bp and 500 bp including all integer values between. For example, a homology arm may be about 2000 bp, about 1500 bp, about 1000 bp, or about 500 bp. In embodiments having two homology arms the homology arms may be the same or different length. Thus, each of the two homology arms may be any length between about 2000 bp and 500 bp including all integer values between. For example each of the two homology arms may be about 2000 bp, about 1500 bp, about 1000 bp, or about 500 bp.

A portion of the vector adjacent to one or both (i.e., between) homology arms modifies the targeted locus in the Spirulina genome by homologous recombination. Techniques for homologous recombination in other organisms are generally known (see, e.g., Kriegler, 1990, Gene transfer and expression: a laboratory manual, Stockton Press). The modification may change a length of the targeted locus including a deletion of nucleotides or addition of nucleotides. The addition or deletion may be of any length. The modification may also change a sequence of the nucleotides in the targeted locus without changing the length. The targeted locus may be any portion of the Spirulina genome including coding regions, non-coding regions, and regulatory sequences. In an embodiment the mutation may delete a gene thereby creating a knock-out organism. In an embodiment the mutation may add a gene that functions as a reporter or marker (e.g., GFP or antibiotic resistance). In an embodiment the mutation may add an exogenous gene. In an embodiment the mutation may add an endogenous gene under control of an exogenous promoter (e.g., strong promoter, inducible promoter, etc.).

A vector for use in the targeted mutagenesis described above may be produced by assembling a vector backbone with an insert sequence. The vector may be created by any known or later developed technique including restriction enzyme digest followed by ligation or Gibson assembly. Gibson assembly may be performed by combining DNA sequences that will become portions of vector backbone with an exonuclease, DNA polymerase, and DNA ligase then incubating at 50° C. for one hour. The vector backbone may be selected for compatibility with the target organism. For Spirulina, suitable vector backbones include, but are not limited to DNA plasmids. The vector backbone may be converted from a continuous loop to a linear form by treatment with an appropriate restriction endonuclease. The ends thereby formed are treated with alkaline phosphatase to remove 5′-phosphate end groups so that the vector may not reform a continuous loop in a DNA ligase reaction without first incorporating an insert segment.

The insert sequence includes one or two homology arms and a nucleotide sequence that, due to differences between the nucleotide sequences of the insert and the wild-type genome sequence of the Spirulina, modify a locus of the Spirulina. The insert sequence includes the one or two flanking regions adjacent to the locus of interest that correspond to the homology arms which are homologous to regions of the Spirulina genome and a portion that is different from the Spirulina genome. The portion of the insert sequence that differs from the wild-type nucleotide sequence of the Spirulina leads to modification of the Spirulina genome due to those differences. The modification may include, but is not limited to, a point mutation, addition of a gene, addition of a regulatory element, addition of a coding region, addition of a non-coding region, deletion of a gene, deletion of a portion of a gene, or deletion of a regulatory element. Generally, it is well-known that strong E. coli promoters work well in Cyanobacteria. If the sequence of the target organism is known, a published sequence may be used to design the insert sequence with homology arm(s). Several strains of Spirulina have been sequenced such as Athrospira plantensis NIES-39. If the sequence is not known a portion of the genome containing the loci of interest may be amplified using PCR and suitable primers. This region may be subsequently sequenced using techniques known to one skilled in the art. In an embodiment an amplified region of the genome may be used to carry out overlap extension PCR to create a deletion. In an embodiment an amplified region of the genome may be digested with a restriction endonuclease that cuts the nucleotide sequence in one place and ligated to an insert nucleotide sequence that has be prepared with compatible ends.

The insert sequence is digested with one or more restriction endonucleases to create ends that are compatible with the ends of the vector backbone. Alternatively, short sequences (e.g., 16-25 bp) that are identical to the terminal regions of the vector backbone are added to the ends of the insert sequence, and these two fragments are circularized by the Gibson assembly method. The length of the insert sequence will be the length of the modifications to the loci of interest and the length of the flanking regions. For example, if the length of the modified sequence is 500 bp and two homology arms each of 2000 bp are desired, then then the entire length of the sequence will be 4500 bp. The polynucleotides sequences used as vectors in the present invention, regardless of the length of the coding sequence itself, may be combined with other sequences, such as promoters, transcriptional terminators, additional restriction enzyme sites, multiple cloning sites, other coding segments, and the like, such that their overall length may vary considerably. It is therefore contemplated that a polynucleotide fragment of almost any length may be employed, with the total length preferably being limited only by the ease of preparation and use in the intended recombinant nucleotide protocol.

### Illustrative Applications

### Neutral Lipid (Wax Ester and Triglyceride) Production

In an embodiment Spirulina may be modified to increase production of neutral lipids, including wax esters and/or triglycerides, beyond the level produced by a wild-type Spirulina grown under the same condition. Triglycerides and wax esters may be used as a feedstock in the production of biofuels and/or various specialty chemicals. For example, triglycerides may be subject to a transesterification reaction, in which an alcohol reacts with triglyceride oils, such as those contained in vegetable oils, animal fats, recycled greases, to produce biodiesels such as fatty acid alkyl esters. Such reactions also produce glycerin as a by-product, which can be purified for use in the pharmaceutical and cosmetic industries. Triglycerides, or triacylglycerols (TAG), consist primarily of glycerol esterified with three fatty acids, and yield more energy upon oxidation than either carbohydrates or proteins. Triglycerides provide an important mechanism of energy storage for most eukaryotic organisms. In mammals, TAGs are synthesized and stored in several cell types, including adipocytes and hepatocytes. In contrast to eukaryotes, the observation of triglyceride production in prokaryotes has been limited to certain actinomycetes, such as members of the genera Mycobacterium, Nocardia, Rhodococcus, and Streptomyces, in addition to certain members of the genus Acinetobacter.

Certain organisms can be utilized as a source of neutral lipids in the production of biofuels. For example, eukaryotic algae naturally produce triglycerides as energy storage molecules, and certain biofuel-related technologies are presently focused on the use of algae as a feedstock for biofuels. Algae are photosynthetic organisms, and the use of triglyceride-producing organisms such as algae provides the ability to produce biodiesel from sunlight, water, CO₂, macronutrients, and micronutrients.

Like algae, cyanobacteria species including Spirulina may obtain energy from photosynthesis, utilizing chlorophyll A and water to reduce CO₂. However, Spirulina lacks the essential enzymes involved in neutral lipid synthesis. Addition of exogenous genes to cyanobacteria increases lipid production. The techniques described above may be used to add genes to Spirulina through targeted, homologous recombination. Thus, the techniques described in this disclosure provide a way to add genes known to increase triglyceride and wax ester production in cyanobacteria such as genes encoding diacylglycerol acyltransferase (DGAT), phosphatidate phosphatase, acetyl-CoA carboxylase (ACCase), aldehyde forming acyl-ACP reductase (AAR), alcohol forming fatty acyl-CoA reductase and alcohol forming fatty acyl-ACP reductase (FAR). The techniques described above may also be used to add portions of genes or polynucleotide sequences encoding polypeptide sequences that differ from wild-type DGAT, phosphatidate phosphatase, and/or ACCase polynucleotide sequences but that still have DGAT enzymatic activity, phosphatidate phosphatase enzymatic activity, and/or ACCase enzymatic activity.

Acyl ACP Reductases (AARs) catalyze the reduction of acyl-ACP's to acyl aldehydes, also known as fatty aldehydes. These enzymes are also known as Fatty Acyl ACP Reductasts (FARs). Fatty aldehydes can serve as a substrate for fatty alcohol biosynthesis by a FAR or long chain alcohol dehydrogenase (ADH). One example of an acyl-ACP reductase is PCC7942_orf1594 from S. elongatus. The family of AAR genes in cyanobacteria (or FAR genes) have been identified using hidden Markov model protein family patterns TIGR045058 (aldehyde forming long chain fatty acyl ACP reductase) and TIGR04059 (long chain fatty aldehyde decarbonylase) from the TIGRRAMs database.

According to one non-limiting theory, certain embodiments may employ AARs in conjuction with FARs or ADHs to increase synthesis of fatty alcohols, which can then be incorporated into wax esters, mainly by the DGAT-expressing (and thus wax ester-producing) photosynthetic microorganisms described herein. Hence, AARs can be used in any of the embodiments described herein, such as those that produce increased levels of free fatty alcohols, where it is desirable to turn these into wax esters. As noted above, these free fatty alcohols can then be esterified to fatty acids (in the form of acyl-ACP) by DGATs to generate wax esters.

Certain embodiments relate to the use of overexpressed AARs to increase synthesis of fatty alcohols, and thereby increase production of wax esters in a wax ester-producing strain (e.g., a DGAT-expressing strain). For instance, certain embodiments may utilize an AAR, in combination with a fatty acyl reductase, and a DGAT. These embodiments may then further utilize an ACP, an ACCase, or both, and/or any of the modifications to glycogen production and storage or glycogen breakdown described herein.

Fatty Acyl Reductases (FAR) catalyze the two step reduction of acyl-ACP's or acyl-COA's to acyl alcohols, also known as fatty alcohols. The first step proceeds via an acyl aldehyde intermediate, which is then converted in a second step to a fatty alcohol. These same enzymes can also directly reduce fatty aldehydes to fatty alcohols (i.e. step two only). In this case they are sometimes referred to as fatty aldehyde reductases. Fatty alcohols can serve as a substrate for wax ester biosynthesis by a DGAT. Many fatty acyl reductases are characterized by three conserved sequence elements. There is an NADPH binding motif, a motif characteristic of the catalytic site of NADP-utilizing enzymes, and a conserved C-terminal domain, referred to as the Male Sterile 2 domain, that is of unknown function.

According to one non-limiting theory, certain embodiments may employ fatty acyl reductases to increase synthesis of fatty alcohols, which can then be incorporated into wax esters, mainly by the DGAT-expressing (and thus wax ester-producing) photosynthetic microorganisms described herein. Hence, fatty acyl reductases can be used in any of the embodiments described herein, such as those that produce increased levels of free fatty alcohols, where it is desirable to turn these into wax esters. As noted above, these free fatty alcohols can then be esterified to fatty acids (in the form of acyl-ACP) by DGATs to generate wax esters.

Certain embodiments relate to the use of overexpressed fatty acyl reductases to increase synthesis of fatty alcohols, and thereby increase production of wax esters in a wax ester-producing strain (e.g., a DGAT-expressing strain). For instance, certain embodiments may utilize a fatty acyl reductase, possibly in combination with an acyl-ACP reductase, and a DGAT. These embodiments may then further utilize an ACP, an ACCase, or both, and/or any of the modifications to glycogen production and storage or glycogen breakdown described herein.

Diacylglycerol acyltransferases (DGATs) are members of the O-acyltransferase superfamily, which esterify either sterols or diacylglycerols in an oleoyl-CoA-dependent manner. DGAT in particular esterifies diacylglycerols, and this esterification represents the final enzymatic step in the production of triacylglycerols in plants, fungi, and mammals. Specifically, DGAT is responsible for transferring an acyl group from acyl-coenzyme-A to the sn-3 position of 1,2-diacylglycerol (DAG) to form triacylglycerol (TAG). The DGAT may be an Acinetobacter baylii ADP1 diacylglycerol acyltransferase (AtfA), a Streptomyces coelicolor DGAT, Plesiomonas Shigelloides DGAT or Alcanivorax borkumensis DGAT. Thus, in an embodiment the DGAT protein comprises a sequence set forth in any one of SEQ ID NOs: 5, 6, or 7. SEQ ID NO: 5 is the sequence of Acinetobacter DGAT. SEQ ID NO: 6: is the sequence of a Streptomyces coelicolor DGAT. SEQ ID NO: 7: is the sequence of Alcanivorax borkumensis DGAT. In plants and fungi, DGAT is associated with the membrane and lipid body fractions. In catalyzing TAGs, DGAT contributes mainly to the storage of carbon used as energy reserves. In animals, however, the role of DGAT is more complex. DGAT not only plays a role in lipoprotein assembly and the regulation of plasma triacylglycerol concentration, but participates as well in the regulation of diacylglycerol levels (Biochemistry of Lipids, Lipoproteins and Membranes 171-203). DGAT proteins may utilize a variety of acyl substrates in a host cell, including fatty acyl-CoA and fatty acyl-ACP molecules. In addition, the acyl substrates acted upon by DGAT enzymes may have varying carbon chain lengths and degrees of saturation, although DGAT may demonstrate preferential activity towards certain molecules.

A “phosphatidate phosphatase” gene as used herein includes any polynucleotide sequence encoding amino acids, such as protein, polypeptide or peptide, obtainable from any cell source, which demonstrates the ability to catalyze the dephosphorylation of phosphatidate (PtdOH) under enzyme reactive conditions, yielding diacylglycerol (DAG) and inorganic phosphate, and further includes any naturally-occurring or non-naturally occurring variants of a phosphatidate phosphatase sequence having such ability. Example phosphatidate phosphatase genes include, but are not limited to, yeast phosphatidate phosphatase, including Saccharomyces cerevisiae phosphatidate phosphatase (yPah1). The Pah1-encoded PAP1 enzyme is found in the cytosolic and membrane fractions of the cell, and its association with the membrane is peripheral in nature. As expected from the multiple forms of PAP1 that have been purified from yeast, pah1Δ mutants still contain PAP1activity, indicating the presence of an additional gene or genes encoding enzymes having PAP1 activity. In an embodiment the phosphatidate phosphatase gene (Pah1) comprises a sequence set forth in SEQ ID NO: 8.

As used herein, an “acetyl COA carboxylase” gene includes any polynucleotide sequence encoding amino acids, such as protein, polypeptide or peptide, obtainable from any cell source, which demonstrates the ability to catalyze the carboxylation of acetyl-CoA to produce malonyl-CoA under enzyme reactive conditions, and further includes any naturally-occurring or non-naturally occurring variants of an acetyl CoA carboxylase sequence having such ability. Acetyl-CoA carboxylase (ACCase) is a biotin-dependent enzyme that catalyzes the irreversible carboxylation of acetyl-CoA to produce malonyl-CoA through its two catalytic activities, biotin carboxylase (BC) and carboxyltransferase (CT). The biotin carboxylase (BC) domain catalyzes the first step of the reaction: the carboxylation of the biotin prosthetic group that is covalently linked to the biotin carboxyl carrier protein (BCCP) domain. In the second step of the reaction, the carboxyltransferase (CT) domain catalyzes the transfer of the carboxyl group from (carboxy) biotin to acetyl-CoA. Formation of malonyl-CoA by acetyl-CoA carboxylase (ACCase) represents the commitment step for fatty acid synthesis, because malonyl-CoA has no metabolic role other than serving as a precursor to fatty acids. Because of this reason, acetyl-CoA carboxylase represents a pivotal enzyme in the synthesis of fatty acids. The ACCase may be a Saccharomyces cerevisiae acetyl-CoA carboxylase (yACC1), a Triticum aestivum ACCase, or a Synechococcus sp. PCC 7002 ACCAse. In an embodiment the ACCase gene comprises a sequence set forth in SEQ ID NO: 9.

Specifically, phosphatidate phosphatase enzymes catalyze the production of diacylglycerol molecules, an immediate pre-cursor to triglycerides, and DGAT enzymes catalyze the final step of triglyceride synthesis by converting the diacylglycerol precursors to triglycerides. Increased intracellular ACCase activity contributes to the increased production of fatty acids because this enzyme catalyzes the “commitment step” of fatty acid synthesis. Specifically, ACCase catalyzes the production of a fatty acid synthesis precursor molecule, malonyl-CoA.

The genes or nucleotides encoding polypeptides with the desired enzymatic activity may be introduced to the Spirulina genome by a vector with a nucleotide sequence encoding DGAT, phosphatidate phosphatase, ACCase, or a polynucleotide sequence that has DGAT enzymatic activity, phosphatidate phosphatase enzymatic activity, or ACCase enzymatic activity between homology arms which target a specific locus of the Spirulina genome. The vector may be any vector suitable for transformation of Spirulina including the vectors described above. The polynucleotides of the vector may be codon-optimized for expression in Spirulina. The vector may include a promoter associated with the added gene. The promoter may be an inducible promoter. As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence. Such nucleotides are typically referred to as “codon-optimized.”

In an embodiment the specific locus is be a gene that is replaced with the nucleotide sequence of the vector. Regulatory elements such as a promoter associated with the replaced gene may be used to direct transcription of the nucleotide sequence from the vector. In an embodiment the specific locus may be a non-coding region of the Spirulina genome. The nucleotide sequence of the vector between the homology arms may include regulatory elements such as a promoter that are introduced into the Spirulina genome together with the genes or nucleotides encoding polypeptides with the desired enzymatic activity. Since wild-type Spirulina do not typically encode the enzymes necessary for triglyceride synthesis the techniques described in this disclosure provide a way to add exogenous genes having DGAT activity, phosphatidate phosphatase activity, and/or ACCase enzymatic activity to Spirulina in order to increase lipid production.

### Reduced Glycogen Production

In an embodiment Spirulina may be modified to accumulate a reduced amount of glycogen as compared to a wild-type Spirulina grown under the same conditions. By reducing the amount of glycogen produced, carbon assimilated by Cyanobacteria is directed to the synthesis of other carbon-based products such as lipids and/or fatty acids. Deletion of glycogen biosynthesis genes in cyanobacteria decrease glycogen accumulation.

By blocking, disrupting, or down-regulating the natural glycogen synthesis and storage pathway, e.g., by gene mutation or deletion, in Cyanobacteria the resulting strains of photosynthetic microorganisms increase carbon flow into other biosynthetic pathways. Examples of other biosynthetic pathways include existing pathways, such as existing lipid biosynthetic pathways, or pathways that are introduced through genetic engineering, such as fatty acid or triglyceride biosynthesis pathways. This modification of deleting a gene associated with glycogen synthesis may be combined with the modification described above to add exogenous genes associated triglyceride synthesis. The techniques described above may be used to delete all or a portion of one or more genes associated with the glycogen synthesis and/or storage pathway in Spirulina including glucose-1-phosphate adenyltransferase (glgC) genes, phosphoglucomutase (pgm) genes, or a glycogen synthase (glgA) genes. Deletions of a portion of a glucose-1-phosphate adenyltransferase (glgC) gene, a phosphoglucomutase (pgm) gene, or a glycogen synthase (glgA) gene that renders the resulting polypeptide without or with a reduced level of enzymatic activity are also contemplated. Decreased glycogen synthesis and/or accumulation may be more pronounced in Spriulina grown under stress conditions such as reduced nitrogen.

Glycogen is a polysaccharide of glucose, which functions as a means of carbon and energy storage in most cells, including animal and bacterial cells. More specifically, glycogen is a very large branched glucose homopolymer containing about 90% α-1,4-glucosidic linkages and 10% α-1,6 linkages. For bacteria in particular, the biosynthesis and storage of glycogen in the form of α-1,4-polyglucans represents an important strategy to cope with transient starvation conditions in the environment.

Glycogen biosynthesis involves the action of several enzymes. For instance, bacterial glycogen biosynthesis occurs generally through the following general steps: (1) formation of glucose-1-phosphate, catalyzed by phosphoglucomutase (Pgm), followed by (2) ADP-glucose synthesis from ATP and glucose 1-phosphate, catalyzed by glucose-1-phosphate adenylyltransferase (GlgC), followed by (3) transfer of the glucosyl moiety from ADP-glucose to a pre-existing α-1,4 glucan primer, catalyzed by glycogen synthase (GlgA). This latter step of glycogen synthesis typically occurs by utilizing ADP-glucose as the glucosyl donor for elongation of the α-1,4-glucosidic chain.

In bacteria, the main regulatory step in glycogen synthesis takes place at the level of ADP-glucose synthesis, or step (2) above, the reaction catalyzed by glucose-1-phosphate adenylyltransferase (GlgC), also known as ADP-glucose pyrophosphorylase. In contrast, the main regulatory step in mammalian glycogen synthesis occurs at the level of glycogen synthase. As shown herein, by altering the regulatory and/or other active components in the glycogen synthesis pathway of photosynthetic microorganisms such as Cyanobacteria, and thereby reducing the biosynthesis and storage of glycogen, the carbon that would have otherwise been stored as glycogen can be utilized to synthesize other carbon-based storage molecules, such as lipids, fatty acids, and triglycerides.

In an embodiment, a Spirulina, expresses a reduced amount of the phosphoglucomutase gene. In particular embodiments, it may comprise a mutation or deletion in the phosphoglucomutase gene, including any of its regulatory elements (e.g., promoters, enhancers, transcription factors, positive or negative regulatory proteins, etc.). Phosphoglucomutase (Pgm), encoded by the gene pgm, catalyzes the reversible transformation of glucose 1-phosphate into glucose 6-phosphate, typically via the enzyme-bound intermediate, glucose 1,6-biphosphate. Although this reaction is reversible, the formation of glucose-6-phosphate is markedly favored.

In an embodiment, a modified Spirulina expresses a reduced amount of a glucose-1-phosphate adenylyltransferase (glgC) gene. In certain embodiments, it may comprise a mutation or deletion in the glgC gene, including any of its regulatory elements. The enzyme encoded by the glgC gene (e.g., EC 2.7.7.27) participates generally in starch, glycogen and sucrose metabolism by catalyzing the following chemical reaction:

ATP+alpha-D-glucose 1-phosphate→diphosphate+ADP-glucose Thus, the two substrates of this enzyme are ATP and alpha-D-glucose 1-phosphate, whereas its two products are diphosphate and ADP-glucose. The glgC-encoded enzyme catalyzes the first committed and rate-limiting step in starch biosynthesis in plants and glycogen biosynthesis in bacteria. It is the enzymatic site for regulation of storage polysaccharide accumulation in plants and bacteria, being allosterically activated or inhibited by metabolites of energy flux.

The enzyme encoded by the glgC gene belongs to a family of transferases, specifically those transferases that transfer phosphorus-containing nucleotide groups (i.e., nucleotidyl-transferases). The systematic name of this enzyme class is typically referred to as ATP: alpha-D-glucose-1-phosphate adenylyltransferase. Other names in common use include ADP glucose pyrophosphorylase, glucose 1-phosphate adenylyltransferase, adenosine diphosphate glucose pyrophosphorylase, adenosine diphosphoglucose pyrophosphorylase, ADP-glucose pyrophosphorylase, ADP-glucose synthase, ADP-glucose synthetase, ADPG pyrophosphorylase, and ADP: alpha-D-glucose-1-phosphate adenylyltransferase.

In an embodiment, Spirulina expresses a reduced amount of a glycogen synthase gene. In particular embodiments, it may comprise a deletion or mutation in the glycogen synthase gene, including any of its regulatory elements. Glycogen synthase (GlgA), also known as UDP-glucose-glycogen glucosyltransferase, is a glycosyltransferase enzyme that catalyses the reaction of UDP-glucose and (1,4-α-D-glucosyl), to yield UDP and (1,4-α-D-glucosyl)ₙ₊₁. Glycogen synthase is an α-retaining glucosyltransferase that uses ADP-glucose to incorporate additional glucose monomers onto the growing glycogen polymer. Essentially, GlgA catalyzes the final step of converting excess glucose residues one by one into a polymeric chain for storage as glycogen.

Classically, glycogen synthases, or α-1,4-glucan synthases, have been divided into two families, animal/fungal glycogen synthases and bacterial/plant starch synthases, according to differences in sequence, sugar donor specificity and regulatory mechanisms. However, detailed sequence analysis, predicted secondary structure comparisons, and threading analysis show that these two families are structurally related and that some domains of animal/fungal synthases were acquired to meet the particular regulatory requirements of those cell types.

Crystal structures have been established for certain bacterial glycogen synthases. These structures show that reported glycogen synthase folds into two Rossmann-fold domains organized as in glycogen phosphorlyase and other glycosyltransferases of the glycosyltransferases superfamily, with a deep fissure between both domains that includes the catalytic center. The core of the N-terminal domain of this glycogen synthase consists of a nine-stranded, predominantly parallel, central β-sheet flanked on both sides by seven α-helices. The C-terminal domain (residues 271-456) shows a similar fold with a six-stranded parallel β-sheet and nine α-helices. The last α-helix of this domain undergoes a kink at position 457-460, with the final 17 residues of the protein (461-477) crossing over to the N-terminal domain and continuing as α-helix, a typical feature of glycosyltransferase enzymes.

These structures also show that the overall fold and the active site architecture of glycogen synthase are remarkably similar to those of glycogen phosphorylase, the latter playing a central role in the mobilization of carbohydrate reserves, indicating a common catalytic mechanism and comparable substrate-binding properties. In contrast to glycogen phosphorylase, however, glycogen synthase has a much wider catalytic cleft, which is predicted to undergo an important interdomain ‘closure’ movement during the catalytic cycle.

Crystal structures have been established for certain GlgA enzymes. These studies show that the N-terminal catalytic domain of GlgA resembles a dinucleotide-binding Rossmann fold and the C-terminal domain adopts a left-handed parallel beta helix that is involved in cooperative allosteric regulation and a unique oligomerization. Also, communication between the regulator-binding sites and the active site involves several distinct regions of the enzyme, including the N-terminus, the glucose-1-phosphate-binding site, and the ATP-binding site.

The glucose-1-phosphate adenyltransferase (glgC), phosphoglucomutase (pgm), and/or glycogen synthase (glgA) genes may be deleted in whole or part using a vector with homology arms that target the upstream and downstream flanking regions of a given gene and recombines with the Spirulina genome to remove nucleotides in the region between the homology arms or replace nucleotides with a different sequence such as a reporter or marker gene. Given the presence of a reporter or marker in the vector sequence, such as a drug-selectable marker, Spirulina cells containing the gene deletion can be readily isolated, identified, and characterized. As one example, selection and isolation may include the use of antibiotic resistant markers known in the art (e.g., kanamycin, spectinomycin, and streptomycin). Such selectable vector-based recombination methods need not be limited to targeting upstream and downstream flanking regions, but may also be targeted to internal sequences within a given gene, as long as that gene is rendered “non-functional.” The deletion of all or part of genes that participate in glycogen synthesis does not harm growth of Spriulina but reduces production of glycogen.

### Modified Carotenoid Production

Carotenoids can be produced from fats and other basic organic metabolic building blocks by Cyanobacteria including Spirulina. Carotenoids function in photosynthesis to protect other components of photosynthetic systems from oxidative stress. Carotenoids may also provide various yellow to red shades of pigmentation. Carotenoid biosynthesis in Cyanobacteria can be modified by reducing the expression of certain genes, increasing the expression of certain genes, and/or introducing exogenous genes.

Cyanobacteria which contains deletions of crtG (2,2′-β-carotene hydroxylase) leads to increased synthesis and accumulation of zeaxanthin while maintaining typical rates of exponential growth. Additionally, an expression of crtR (3,3′-β-carotene hydroxylase) or crtZ (cartonenoid-3,3′-hydroxylase) has been demonstrated to lead to production of zeaxanthin in the E. coli strain, where a series of genes involved in β-carotene synthesis are introduced, crtE, crtB, crtI and crtY. In an embodiment the crtR gene comprises a sequence set forth in SEQ ID NO: 10. In an embodiment the crtZ gene is from Brevundimonas sp. SD212 and comprises a sequence set forth in SEQ ID NO: 11.

Additionally, introduction of crtW (β-carotene oxygenase) and crtZ (cartonenoid-3,3′-hydroxylase) leads to synthesis and accumulation of astaxanthin and canthaxanthin in Cyanobacteria. In an embodiment the crtW gene is from Brevundimonas sp. SD212 and comprises a sequence set forth in SEQ ID NO: 12.

The techniques described in this disclosure may be used to knock out or render non-functional Spirulina genes in for crtG. Additionally the techniques described in this disclosure may be used to add crtR, crtZ, and crtW genes to the Spirulina genome under control of a native Spirulina promoter or under control of an exogenous promoter including strong promoters and inducible/repressible promoters. In an embodiment, a promoter regulating expression of crtW and crtZ genes may be a tightly regulated promoter such that in the absence of induction there is no or essentially no expression of the exogenous genes.

For example, to produce carotenoids, a modified Spirulina may contain an overexpressed carotenoid hydroxylase (e.g., β-carotene hydroxylase). In these and related embodiments, carotenoid production can be further increased by subjecting the modified photosynthetic microorganism to a stress condition such as, but not limited to, nitrogen deprivation. One illustrative carotenoid hydroxylase is encoded by crtR of A. plantensis NIES39_R00430. Another illustrative carotenoid hydroxylase is encoded by crtZ of Pantoea ananatis. Also included are homologs or paralogs thereof, functional equivalents thereof, and fragments or variants thereofs. Functional equivalents can include carotenoid hydroxylase with the ability to add hydroxyl groups to β-carotene. These and related embodiments can be further combined with reduced expression and/or activity of an endogenous gylcogen-pathway gene (e.g., glgC in A. plantensis), described herein, to shunt carbon away from glycogen production and towards carotenoids.

Modified Phycocyanin and/or Phycoerythrin Production

In an embodiment Spirulina may be modified to increase production of phycocyanin and/or phycoerythrin beyond the level produced by a wild-type Spirulina grown under the same condition. Phycocyanin and phycoerythrin associated with their bilin chromophores, are natural occurring blue and red pigments, respectively, that are used in the cosmetic, food, and medical imaging industries. These pigment molecules can be purified from cyanobacteria.

One distinguishing feature of many cyanobacteria is a massive light harvesting structure called the phycobilisome. The phycobilisome, which give cyanobacteria their diversity of colors, accounts for up to 60% of the cell's protein. These massive protein structures transfer energy directly to the photosynthetic reaction centers for photochemistry.

The pigment molecules in phycobilisomes that absorb light for photochemistry are linear teterapyrrole molecules called bilins. All bilins are covalently bound through a cysteine thioether linkage to phycobiliproteins. There are three major classes of phycobiliproteins in phycobilisomes; allophycocyanin (AP), phycocyanin (PC) and phycoerythrin (PE). Each class contains α and β subunits that form heterodimers. Both the α and β subunits of the heterodimer can bind one or more of the bilin pigments.

The major structural components that comprise the phycobilisome can be subdivided into two groups; the “core” substructure and the “rod” substructure. In Synechococcus sp. strains, the core substructure consists of two cylinders made of a few types of phycobiliproteins and core linker proteins. The rod substructures are composed of hexamers of the PC α and PC β heterodimer. An average of three of these heterohexamers are connected via the linker proteins, and stacked on the core substructure.

The techniques described above may be used to add genes to Spirulina through targeted, homologous recombination. The techniques described in this disclosure provide a way to add genes known to encode phycocyanin, or phycoerythrin. The techniques described above may also be used to add portions of genes or polynucleotide sequences encoding polypeptide sequences that differ from wild-type phycocyanin, or phycoerythrin, but that still have the ability to associate with a bilin chromophore.

### EXAMPLES

Certain embodiments of the present disclosure now will be illustrated by the following Examples. The present disclosure may, however, be embodied in many different forms and should not be construed as limited to the embodiments set forth herein; rather, these embodiments are provided so that this disclosure will be thorough and complete, and will fully convey the scope of the disclosure to those skilled in the art.

### Example 1

Athrospira plantensis NIES-39 cells obtained from the Microbial Culture Collection at the National Institute for Environmental Studies (Japan) were cultured in SOT medium with shaking under continuous illumination. When the optical density at 750 nm reached 0.6-0.8, cells were harvested at 1,500×g for 10 min from 60 ml of culture and resuspended in 30 ml of a pH balancer and salt solution. Cells were collected by centrifugation under the same conditions and washed three times with a polyether osmotic stabilizer. The cell suspension was twice diluted in the osmotic stabilizer and then spun down at 1,500×g for 5 min and a total volume of the cell suspension was adjusted to 2 mL.

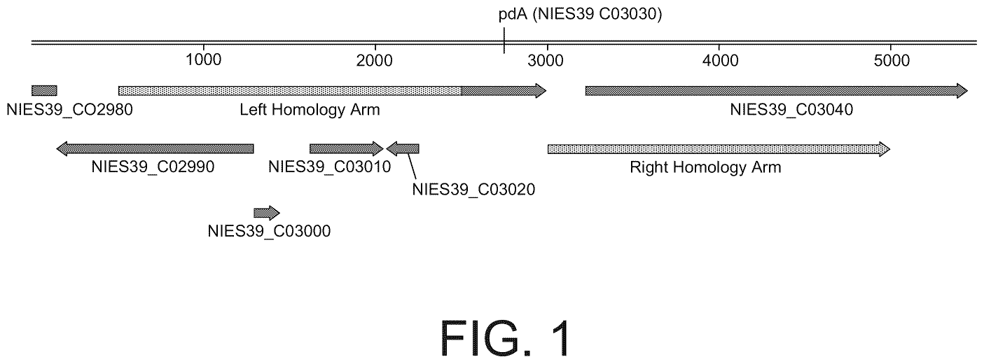

FIG. 1 shows a targeted gene locus of A. plantensis. This gene locus includes the pilA gene (encoding pili protein). Regions of the A. plantensis genome corresponding to the left homology arm and the right homology arm are also shown. The left homology arm is adjacent to the “left” end (5′-end) of the gene locus of interest and the right homology arm is adjacent to the “right” end (3′-end) of the gene locus of interest. In this example the left homology arm and the right homology arm are about 2000 bp long.

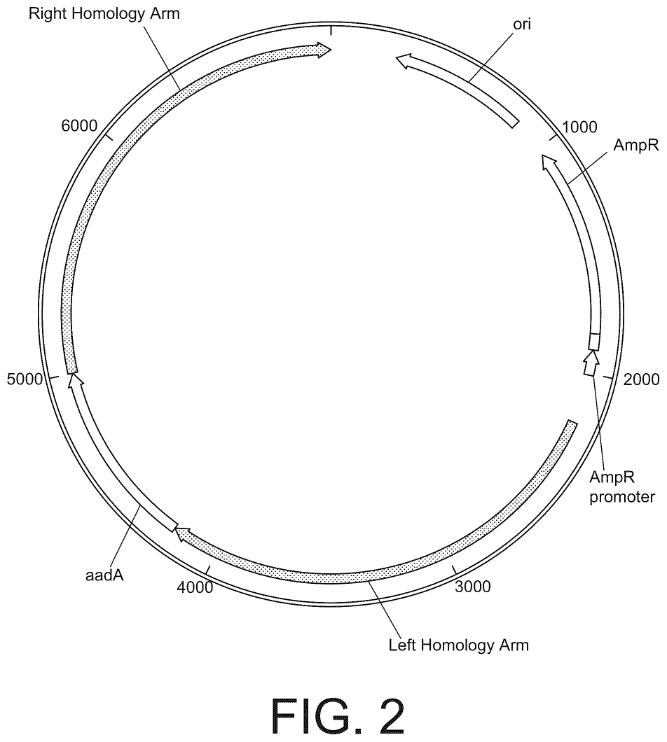

FIG. 2 shows the pApl-pilA/aadA plasmid generated by assembling four PCR fragments containing a vector backbone, the left homology arm, the right homology arm and the aminoglycoside adenlytransferase gene (aadA) using the Gibson assembly method. This plasmid was then used to transform A. plantensis. The pApl-pilA/aadA plasmid includes aadA placed between the homology arms. aadA confers resistance to spectinomycin and streptomycin. Thus, homologous recombination with this plasmid replaces the pilA gene with the aadA gene. The plasmid also contains the AmpR gene (ampicillin resistance) under control of an AmpR promoter and an origin of replication (ori) in E. coli. 2.5 μg of this plasmid was mixed with 200 μL of the cell suspension. Immediately following mixing with the plasmid, the cells were electroporated using a Gene Pulser Xcell™ Microbial System (Bio-Rad, USA).

Following electroporation cells were grown without shaking at reduced illumination in SOT medium for 48 h then transferred to SOT medium supplemented with streptomycin. Presence of streptomycin in the medium selected for transformed cells. The medium including streptomycin was replaced every few days to maintain efficacy of the antibiotic. After growth in the streptomycin medium, the cells were cultured in SOT medium with streptomycin and spectinomycin.

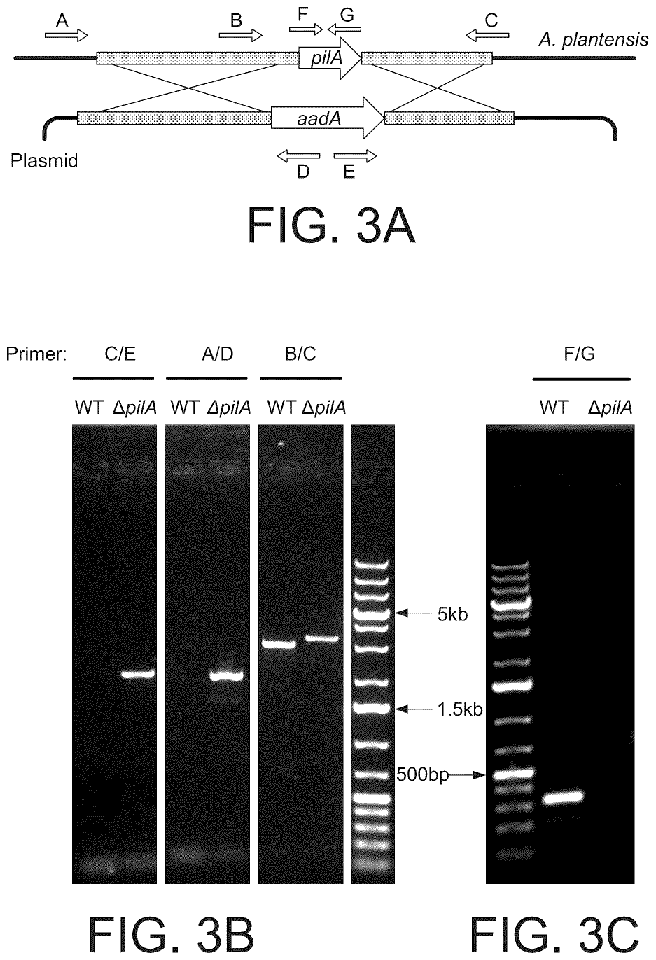

FIG. 3A is a schematic of substitution of the open reading frame of pilA gene in A. plantensis with the aminoglycoside adenylyltransferase (aadA) gene contained in the pApl-pilA/aadA plasmid. Following successful homologous recombination the pilA gene was replaced with the aadA gene. Homology arms are shown as thick lines in the schematic. Three primers A, B, and C were identified that bind to the A. plantensis genome and two primers D and E were identified that bind to the aadA gene in the plasmid. The primers A and C were designed to initiate DNA synthesis by a DNA polymerase outside of the homology arms that exist on the aadA-containing plasmid. Thus, primers A and C bind to regions of the A. plantensis genome that remain unchanged following transformation. Primer A has SEQ ID NO: 13. Primer C has SEQ ID NO: 14. Primer B binds to the homology arm upstream of the pilA gene. Primer B has SEQ ID NO: 15. Primers D and E bind to the upstream and downstream ends, respectively, of the aadA gene. Primer D has SEQ ID NO: 16. Primer E has SEQ ID NO: 17.

FIG. 3B shows results of polymerase chain reaction (PCR) conducted with combinations of different primer pairs comparing wild-type A. plantensis (WT) and A. plantensis following transformation with the pApl-pilA/aadA plasmid (ΔpilA) as described above. Supernatants of boiled bacteria harvested after growth in streptomycin and spectinomycin were used as PCR templates. PCR was conducted under the following conditions: initial denaturation at 98° C. for 1 min and 35 cycles of denaturation at 98° C. for 30 sec, annealing at 71° C.-0.1° C. per cycle for 15 sec and extension at 72° C. for 1.5 min, followed by final extension at 72° C. for 2 min then cooling down to 4° C., for a reaction mixture with primers B and C; initial denaturation at 98° C. for 1 min, followed by 35 cycles of denaturation at 98° C. for 30 sec, annealing at 66° C. for 15 sec and extension at 72° C. for 1 min, followed by final extension at 72° C. for 2 min and cooling down to 4° C., for reaction mixtures with the rest of primers. The integration of aadA into the targeted locus was confirmed using two pairs of the primers (C/E and A/D). The ladder run on the gel has prominent bands at 5 kb, 1.5 kb, and 500 bp. Amplification using the C/E primer pair was not detected on WT but did yield a strong band at 2.2 kb on ΔpilA. Amplification with the A/D primer pair was similarly was not detected on WT but did yield a strong band at 2.1 kb on ΔpilA. Use of the primers B and C allows PCR amplification of the genomic sequences spanning either of pilA or aadA. Amplification of the WT sample (containing pilA) generated a slightly shorter PCR product of about 3.1 kb than amplification of the transformed sample ΔpilA (containing aadA) of about 3.4 kb. This confirms expectations because the aadA sequence is about 300-bp longer than the pilA sequence. The PCR results demonstrate that the endogenous pilA gene was replaced with the aadA gene.

FIG. 3C shows that PCR with primers F and G complementary to the pilA gene failed to amplify any DNA from transformed cells grown in SOT medium supplemented with streptomycin and spectinomycin indicating that every copy of the pilA gene was replaced in cultures grown in the presence of the antibiotics. However, amplification with primers F and G amplified a 340-bp sequence from WT grown in SOT medium without antibiotics confirming that primers F and G are effective at amplifying the pilA gene. Primer F has SEQ ID NO: 18. Primer G has SEQ ID NO: 19.The ladder run on this gel has prominent bands at 5 kb, 1.5 kb, and 500 bp.

### Example 2

Arthrospira sp. PCC 8005 cells were obtained from the Pasture Culture Collection (PCC) of Cyanobacteria maintained in the Laboratory Collection of Cyanobacteria, at Pasteur Institut (Paris) and cultured in SOT medium with shaking under continuous illumination for three days. The cells were diluted by 3 or 4 times in fresh SOT medium during the three day period. The cells were maintained under the above conditions until the optical density at 750 nm reached a desired level. Cells were then harvested at 3,200×g for 10 min from the culture. The supernatant was removed and the cells were suspended in a pH balancer and salt solution to neutralize pH. After centrifugation under the same conditions, cells were washed with 1-50 ml of 0.1-100 mM NaCL and an osmotic stabilizer three times, and then transferred into two 1.5 ml polypropylene microcentrifuge tubes (e.g., Eppendorf® tubes). The 1.5 ml microcentrifuge tubes were centrifuged at 3,200×g for 5 min at room temperature, and the supernatant was discarded. The cell suspension were twice diluted in NaCL and osmolyte solution then centrifuged again at 3,200×g for 5 min. The supernatant was removed and a total volume of the cell suspension was adjusted to 450 μl.

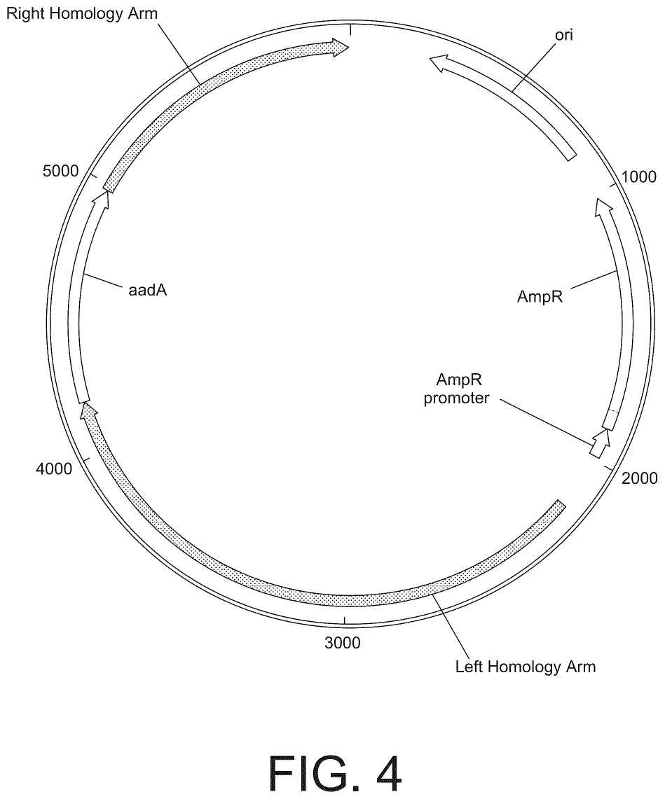

FIG. 4 shows the pApl-pilA8005/aadA plasmid (SEQ ID NO: 2) generated by using the Gibson assembly method to join four PCR fragments containing a vector backbone, a left homology arm, a right homology arm, and aadA encoding aminoglycoside adenlytransferase. In the pApl-pilA8005/aadA plasmid, aadA is located between the right and left homology arms which contain sequences identical to the genomic sequences found upstream and downstream of the pilA open reading frame in Arthrospira sp. PCC 8005. The left homology arm is 2.0 kb and the right homology arm is 1.0 kb. aadA confers resistance to spectinomycin and streptomycin. Thus, homologous recombination between Arthrospira sp. PCC 8005 and this plasmid replaces the pilA gene of Arthrospira sp. PCC 8005 with the aadA gene. The pApl-pilA8005/aadA plasmid also contains an operon encoding β-lactamase (AmpR) which confers ampicillin resistance under control of an AmpR promoter and an origin of replication (ori) from E. coli.

Four hundred μL of the suspended cells were mixed with 0.1-20 μg of the pApl-pilA8005/aadA plasmid, and transferred into an electroporation cuvette. Immediately following mixing, electroporation was performed using a Gene Pulser Xcell™ Microbial System (Bio-Rad, USA) electroporation system.

Following electroporation, 1 ml of SOT medium was added into the cuvette then the cells and SOT medium were transferred into a 50 mL polypropylene conical centrifuge tube (e.g., Falcon® tube) containing 9 mL of SOT medium. Cells were incubated at reduced illumination at 30° C. Successful transformants identified by antibiotic resistance were selected in the presence of streptomycin and spectinomycin.

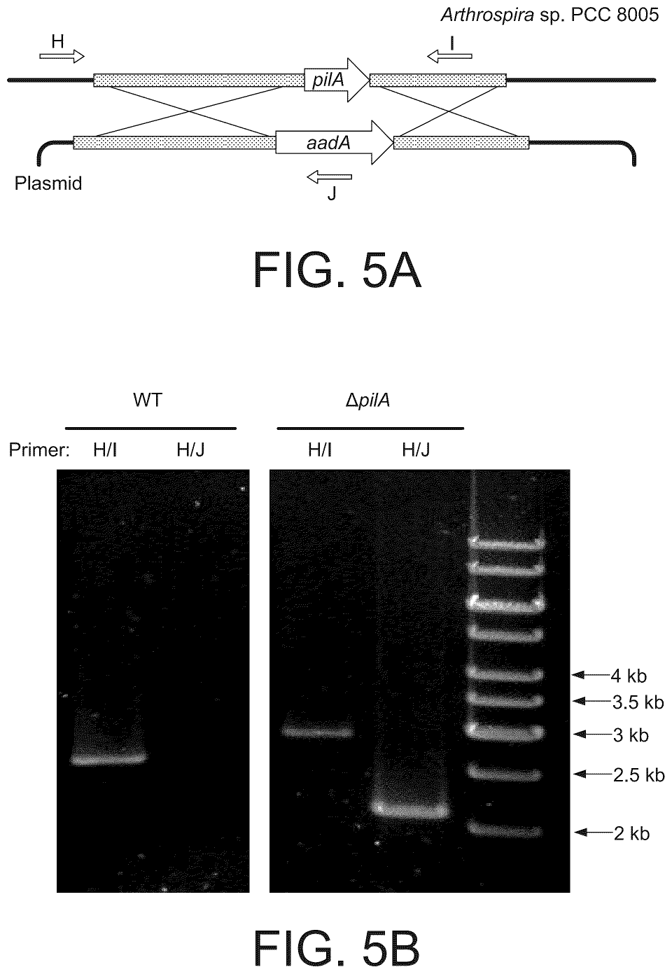

FIG. 5A is a schematic of substitution of the open reading frame of pilA gene in Arthrospira sp. PCC 8005 with the aminoglycoside adenylyltransferase (aadA) gene contained in the pApl-pilA8005/aadA plasmid. Following successful homologous recombination the pilA gene was replaced with the aadA gene. Homology arms are shown as shaded boxes in the schematic. Two primers H and I were identified that bind to the Arthrospira sp. PCC 8005 genome and one primer J was identified that binds to the aadA gene in the plasmid. Primer H initiates DNA synthesis by a DNA polymerase outside of the homology arms that exist on the pApl-pilA8005/aadA plasmid. Thus, primer H binds to a region of the Arthrospira sp. PCC 8005 genome that remains unchanged following transformation. Primer H has SEQ ID NO: 20. Primer I binds to the homology arm upstream of the pilA gene. Primer I has SEQ ID NO: 21. Primer J has SEQ ID NO: 22.

FIG. 5B shows results of PCR conducted on DNA extracted from the cells cultured in the presence of the antibiotics. The PCR was performed with primer pairs H/I and H/J on wild-type Arthrospira sp. PCC 8005 (WT) and Arthrospira sp. PCC 8005 following transformation with the pApl-pilA8005/aadA plasmid resulting in deletion of pilA (ΔpilA). PCR was conducted under the conditions described above in Example 1. Because there is no annealing site for primer J in wild-type Arthrospira sp. PCC 8005, the H/J primer pair will result in amplification only in the reaction mixtures containing DNA extracted from the transformed cells, indicating that pilA of Arthrospira sp. PCC 8005 was replaced with aadA. The integration of aadA into the targeted locus was confirmed using two pairs of the primers (H/I and H/J) and comparing the results by running the PCR products on a 1% w/v agarose gel.

Amplification of DNA extracted from wild-type Arthrospira sp. PCC 8005 with primer pair H/I shows a band between 2.5 kb and 3 kb indicating presence of the pilA gene. As expected, amplification of WT Arthrospira sp. PCC 8005 with the H/J primer pair does not result in any band because without transformation aadA is not present in the Arthrospira sp. PCC 8005 genome. DNA extracted from the transformed cells resulted in PCR products when amplified with either the H/I primer pair or the H/J primer pair. When the H/I primers are used on DNA from transformed cells, a band is present at approximately 3 kb which is slightly larger than the band resulting from H/I primer pair amplification of wild-type Arthrospira sp. PCC 8005. This demonstrates that the chromosome containing aadA was fully segregated since aadA is approximately 300 bp longer than pilA. The transformed cells, unlike the WT cells, also amplified with the H/J primer pair yielding a band slightly above 2 kb. The PCR results demonstrate that the endogenous pilA gene was replaced with the aadA gene and that the aadA gene was duplicated on all chromosomal copies.

### Example 3

Athrospira plantensis NIES-39 was modified using the procedure from Example 2 to introduce exogenous genes encoding β-carotene 3,3′-hydroxylase (CrtZ) and β-carotene 4,4′-ketolase (CrtW) into the endogenous NIES39_Q01220 locus. A. plantentis does not naturally synthesize astaxanthin, but introduction of crtZ and crtW provides a biosynthesis pathway enabling the synthesis of this valuable carotenoid. The crtZ gene was obtained from Pantoea ananatis (SEQ ID NO: 23) and the crtW gene was obtained from Brevundimonas sp. SD212 (SEQ ID NO: 24). Both genes have been verified to function in Synechococcus elongatus PCC7942 and were codon optimized for expression in cyanobacteria.

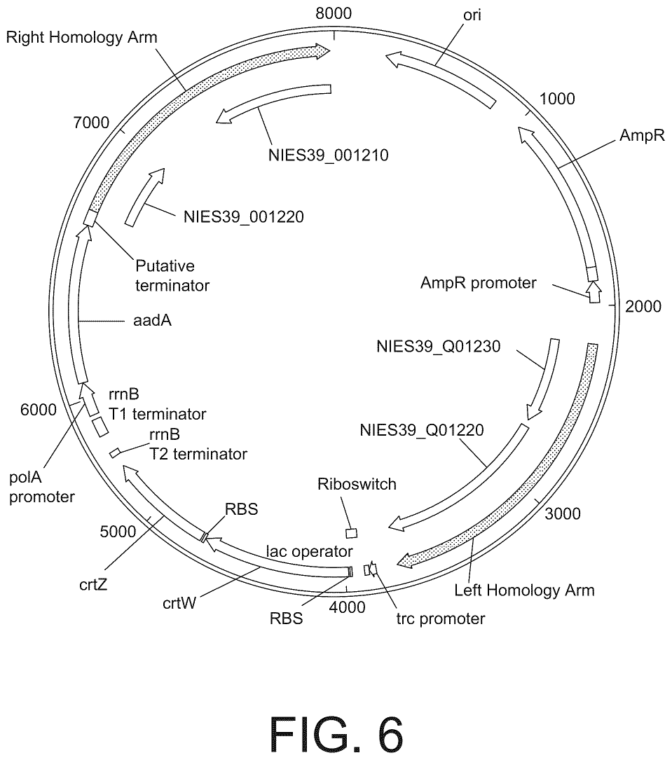

A pApl-NS1/Prs-crtW-crtZ plasmid was created to introduce crtZ and crtW into A. plantentis. This plasmid includes the aadA gene and the crtZ and crtW genes downstream of an inducible trc promoter connected to a riboswitch. FIG. 6 shows the design of the pApl-NS1/Prs-crtW-crtZ plasmid (SEQ ID NO: 3). This plasmid was generated by the same techniques described in Example 1.

Electroporation and culturing techniques were initially performed as described in Example 1 but for using the pApl-NS1/Prs-crtW-crtZ plasmid instead of the pApl-pilA/aadA plasmid from Example 1. Following this change transformation was not successful. Without being bound by theory expression of CrtW and CrtZ in cyanobacteria is believed to disrupt photosystems which may inhibit cell growth. It is theorized that the selected promoter, absent induction, did not sufficiently repress expression of the crtZ and crtW genes. Thus, presence of CrtW and CrtZ proteins may have prevented any transformed cells from growing. Transformation with genes that can inhibit growth may be dependent on a promoter design that very tightly regulates gene expression. Interaction between promotors, inserted genes, and transformation success is an area of further study.

### Example 4

Athrospira plantensis NIES-39 is modified by adding additional copies of the cpcA and cpcB genes encoding C-phycocyanin α and β subunits. WT Athrospira plantensis NIES-39 has only a single set of these genes. Increasing the copy number of these endogenous genes increases synthesis of blue-colored C-phycocyanin. The cpcA (SEQ ID NO: 25) and cpcB (SEQ ID NO: 26) genes were obtained from WT Athrospira plantensis NIES-39.

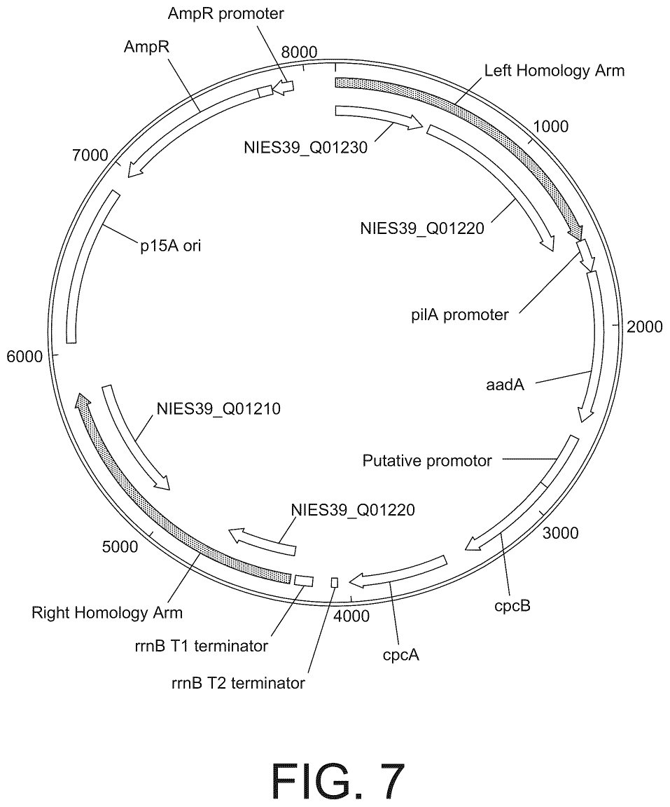

The plasmid used to introduce cpcB and cpcA into A. plantentis was created by the techniques described in Example 1. FIG. 7 shows a schematic of the pApl-NS1/aadA-cpcBA plasmid (SEQ ID NO: 4). This plasmid contains a low-copy p15A origin of replication, the pilA promoter (a 168-bp sequence upstream of the pilA open reading frame), aadA, and the genomic sequence (SEQ ID NO: 27) ranging from 278-bp upstream of cpcB to the 3′ end of cpcA in the middle of gene locus NIES39_Q01220. The 1.5-kb flanking regions of the insertion site are the left and right homology arms that integrate the exogenous gene operons into the targeted gene locus (i.e. NIES39_Q01220).

Athrospira plantensis NIES-39 was transformed with the pApl-NS1/aadA-cpcBA plasmid using the protocol we described in Example 3.

Following transformation, the strain carrying the second copies of cpcB and cpcA was grown in 160 mL of SOT medium in under the same conditions as its parental strain (i.e. Arthrospira platensis NIES-39). Cells were harvested from 12 mL of each culture every other day to quantify the amounts of C-phycocyanin and allophycocyanin. The collected cells were resuspended in 5 ml of 100 mM sodium phosphate buffer (pH 6.0), and centrifuged at 3,200×g for 5 min at room temperature.

The pellets were suspended in 2.5 mL of 100 mM sodium phosphate buffer (pH 6.0); and 700 μl of the suspended cells were transferred on pre-scaled PVDF (polyvinylidene fluoride or polyvinylidene difluoride) membranes. The cells were extensively washed with water (15-20 mL×2) on the membranes, and dried in an oven overnight in order to calculate the weight of dry biomass. The remaining cells suspended in 100 mM sodium phosphate buffer (pH 6.0) were stored at −80° C. and diluted in the same 100 mM sodium phosphate buffer to 2.0 mg dry cell weight per mL of buffer. The cells were lysed by gentle agitation at 30° C. overnight and centrifuged at 16,000×g for 30 min at 4° C. The supernatant was transferred into new tubes, and centrifuged again under the same conditions. The supernatant was diluted in 100 mM sodium phosphate buffer (pH 6.0) and the optical densities were measured between 450 nm and 750 nm in 5-nm intervals to confirm that chlorophyll and carotenoids were excluded. The contents of C-phycocyanin (Cpc) and allophycocyanin (Apc) were calculated using the optical densities of clarified cell lysates at 620 nm and 650 nm. Concentrations of C-phycocyanin and allophycocyanin were calculated using the equation OD_{λ}=C_{Cpc}×E_{λ}, Cpc+C_{Apc}×E_{λ,Apc} in which λ equals 620 or 650 nm, where C_{Cpc} is the concentration of C-phycocyanin (mg/mL), E_{λ,Cpc} is the extinction coefficient of C-phycocyanin, C_{Apc} is the concentration of allophycocyanin (mg/ml), E_{λ,Apc} is the extinction coefficient of allophycocyanin, and OD (optical density) is the absorbance. Use of this equation is described in Yoshikawa and Belay (2008) J. AOAC Int.

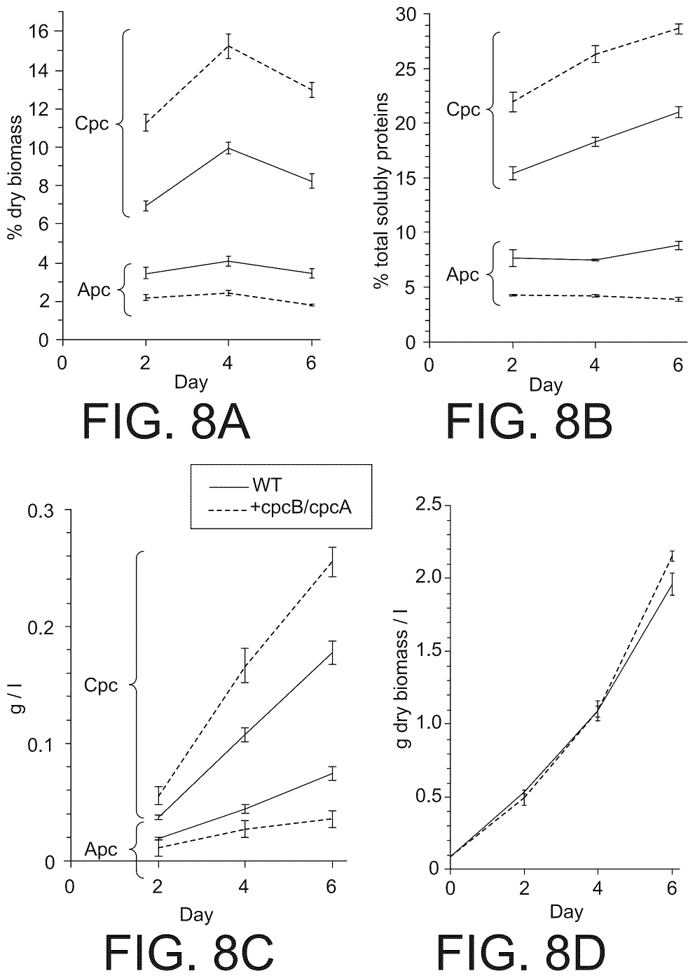

FIGS. 8A-D are graphs comparing phycocyanin production and growth of Arthrospira platensis NIES-39 transformed to include a total of two copies of each of cpcA and cpcB (dotted lines) and WT Arthrospira platensis NIES-39 (solid lines). Error bars for all graphs show standard deviation from three independent experiments. Cells were harvested from the cultures every other day i.e. on days 2, 4, and 6 as described above. The data for all FIGS. 8A-D were gathered from cells grown under the same conditions.

FIG. 8A shows Cpc and Apc production measured as percent of dry biomass. The transformed strain (dotted line) had approximately 3-5% more of its biomass as Cpc than the WT strain (solid line). For both strains the percentage of Cpc peaked on day 4 then decrease. Levels of Apc were about 1% lower in the transformed strain than in the WT strain.

Table 1 shows phycocyanin levels as a percentage of dry biomass further analyzed as pure phycocyanin and crude phycocyanin.

TABLE 1
% dry biomass

| | | | pApl-NS1/aadA-cpcBA |
|---|---|---|---|
| | Day | WT | transformed strain |
| Pure | 2 | 8.68 ± 0.372 | 12.2 ± 0.490 |
| Phycocyanin | 4 | 12.0 ± 0.406 | 16.3 ± 0.685 |
| (%) | 6 | 9.96 ± 0.476 | 13.7 ± 0.391 |
| Crude | 2 | 18.6 ± 0.802 | 26.3 ± 1.06 |
| Phycocyanin | 4 | 25.8 ± 0.875 | 35.1 ± 1.48 |
| (%) | 6 | 21.4 ± 1.02 | 29.6 ± 0.843 |

Pure phycocyanin was calculated using the equation:

$$\% of dry bio mass as pure phycocyanin = 100 ( \frac{{OD}_{260} \times dilution factor}{7.3 \times dry weight} ) .$$

Crude phycocyanin represents all phycobiliprotein pigments and was calculated using the equation:

$$\% of dry bio mass as crude phycocyanin = 100 ( \frac{{OD}_{260} \times dilution factor}{3.39 \times dry weight} ) .$$

FIG. 8B shows Cpc and Apc production measured as percent of total soluble proteins. For both the WT (solid line) and transformed (dotted line) strains the percent of Cpc increased with time. The transformed strain had approximately 5% more of it total soluble proteins as Cpc than the WT strain. Levels of Apc were lower and relatively stable for both strains with the transformed strain having about 1% fewer of total soluble proteins as Apc than the WT strain.

FIG. 8C shows total Cpc and Apc production in grams of phycocyanin per liter of cell culture. The total Cpc production of the transformed strain (dotted line) was more than that of the WT strain (solid line) at all time points and the difference increased with time. Apc levels also increased with time, but the WT strain produced higher levels of Apc than the transformed strain. The contents of Cpc and Apc varied depending on the growth phase, but at all time points and under all conditions, the transformed strain accumulated 50-100% greater amounts of Cpc and less Apc than the WT strain.

FIG. 8D compares growth of the WT and transformed strains. The transformed strain grew (dotted line) at the same rate as the WT strain (solid line) on days 0-4 and only slightly slower on day 6.

### Example 5

To analyze extracts from WT Arthrospira platensis NIES-39 and the transformed strain described above, both strains were cultured in 50 L photobioreactors of SOT medium to obtain biomass enough for protein extraction. The cultures were concentrated using a filter cartridge then the cells were harvested by centrifugation at 3,000×g for 10 min. The concentrated cells were then lyophilized. Two grams of dried biomass from each of the two strains were suspended in 20 ml of water and centrifuged at 3,200×g for 5 min to collect swelling biomass. The supernatant was removed and the pellet was suspended in 20 mL of water. After centrifugation under the same conditions, the pellets were then suspended in 80 mL of 20 mM sodium phosphate buffer (pH 6.0) by repeated pipetting and gently stirred at room temperature for 30 min. Sonication was performed twice at 50% of the maximal amplitude for 30 sec at room temperature using a Qsonica sonicator Q700. Following sonication, the suspended cells were stirred continually overnight at room temperature. To recover water-soluble extracts from the cell lysates, the samples were centrifuged at 4° C. for 30 min at 10,000×g once, then at 16,000×g twice. Seventy two milliliter of the supernatant from each cell lysate was transferred into a glass beaker, and 45 mL of 100% saturated ammonium sulfate in 20 mM sodium phosphate (pH 6.5) was gradually dropped into the beaker where the clarified cell extract was gently stirred at 4° C. The mixtures of the cell lysates and ammonium sulfate were stirred for an hour, precipitated proteins were recovered by centrifugation at 16,000×g for 30 min at 4° C. The pellet was suspended with 20 mM sodium phosphate buffer (pH 6.0) to adjust the total volume to 50 ml, and dialyzed using Slide-A-Lyzer cassettes against 2 L of 20 mM sodium phosphate buffer (pH 6.0) at 4° C. The outer buffer was exchanged three times (3 hours, overnight, then 4 hours). The samples were centrifuged at 4° C. for 30 min at 16,000×g, and the recovered supernatant were centrifuged again under the same conditions.

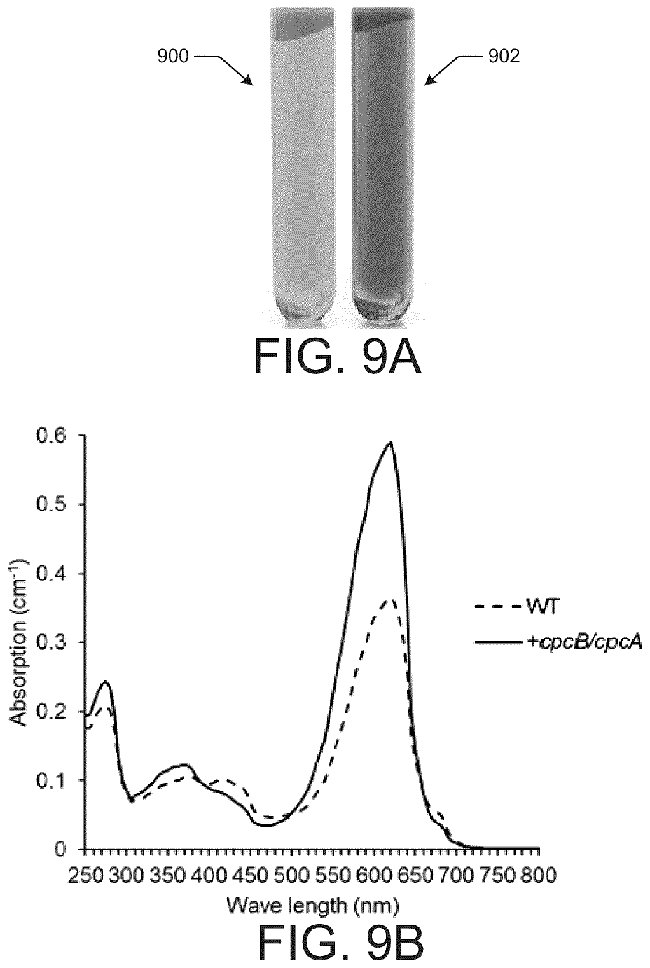

FIG. 9A is a photograph showing that supernatants recovered from the final centrifugation of the two strains have different intensity of blue color. The tube 900 containing WT Arthrospira platensis NIES-39 has a lighter blue color; and the tube 902 containing the strain transformed with the pApl-NS1/aadA-cpcBA plasmid has a darker blue color. C-phycocyanin has an intense blue color with a single visible absorbance maximum around 620 nm. Thus, the deeper blue color was expected from the supernatant of the C-phycocyanin-overproducing strain.

FIG. 9B is a graph showing absorption differences between the transformed strain (solid line) and the WT strain (dotted line). Both strains exhibited an absorbance peak around 620 nm. The peak absorbance for the transformed strain was approximately 0.60 cm⁻¹ and the peak for the WT strain was approximately 0.35 cm⁻¹. This quantitatively confirms the qualitative color difference shown in FIG. 9A. Quality of C-phycocyanin extract can be determined by the ratio of the optical density at 620 nm (OD₆₂₀) to the optical density at 280 nm (OD₂₈₀). A higher ratio indicates a higher yield and purity of C-phycocyanin that may be suitable for higher-value uses such as cosmetics rather than lower value uses such as food grade dye. The graph in FIG. 9B shows that the OD₆₂₀/OD₂₈₀ ratio for the transformed strain is 2.5 compared to 1.8 for the WT strain. This indicates a 40% higher concentration of C-phycocyanin from the recovered supernatant of the transformed strain.

### ILLUSTRATIVE EMBODIMENTS

Embodiment 1 is a Spirulina comprising at least one stable, targeted mutation.

Embodiment 2 is the Spirulina of any one of embodiments 1, 4-11, or 25, wherein the Spirulina is Arthrospira platensis or Arthrospira maxima.

Embodiment 3 is the Spirulina of any one of embodiments 1, 2, 4-11, or 25, wherein the Spirulina is Arthrospira platensis NIES-39 or Arthrospira sp. PCC 8005.

Embodiment 4 is the Spirulina of any one of embodiments 1-3, 5-11, or 25, wherein the stable targeted mutation is inherited for at least 10 generations.

Embodiment 5 is the Spirulina of any one of embodiments 1-4, 6-11, or 25, wherein the stable targeted mutation is integrated into a chromosome of the Spirulina.

Embodiment 6 is the Spirulina of any one of embodiments 1-5, 7-11, or 25, wherein the targeted mutation comprises a deletion or disruption of at least a portion of a gene.

Embodiment 7 is the Spirulina of any one of embodiments 1-6 or 8-11 wherein the targeted mutation comprises an addition of an additional copy of an endogenous gene or addition of an exogenous gene.

Embodiment 8 is the Spirulina of any one of embodiments 1-7, 10, or 11 wherein the targeted mutation comprises addition of a gene regulatory element.

Embodiment 9 is the Spirulina of embodiment 8, wherein the gene regulatory element is a promoter, a regulatory protein binding site, a RNA binding site, a ribosome binding site, or a RNA stability element.

Embodiment 10 is the Spirulina of any one of embodiments 1-9 or 11 wherein the targeted mutation comprises addition of an exogenous or endogenous protein domain.

Embodiment 11 is the Spirulina of embodiment 10, wherein the exogenous protein domain is a phosphorylation site, a stability domain, a protein targeting domain, or protein-protein interaction domain.

Embodiment 12 is a method of creating a targeted mutation in Spirulina, the method comprising: contacting the Spirulina with an osmotic stabilizer; contacting the Spirulina with a vector having homology arms; and inducing artificial competence in the Spirulina.

Embodiment 13 is the method of any one of embodiments 12 or 15-24, wherein the Spirulina is Spirulina platensis.

Embodiment 14 is the method of any one of embodiments 12 or 15-24, wherein the Spirulina is Arthrospira platensis NIES-39 or Arthrospira sp. PCC 8005.

Embodiment 15 is the method of any one of embodiments 12-24, wherein the osmotic stabilizer is at least one of polyethylene glycol, ethylene glycol, glycerol, glucose, or sucrose.

Embodiment 16 is the method of any one of embodiments 12-24, wherein the vector is a DNA vector, a linear vector, a circular vector, a single stranded polynucleotide, or a double stranded polynucleotide.

Embodiment 17 is the method of any one of embodiments 12-24, wherein the vector comprises a vector having SEQ ID NO: 1, SEQ ID NO: 2, or SEQ ID NO: 4.

Embodiment 18 is the method of any one of embodiments 12-24, wherein at least one of the homology arms is at least 500 bp.

Embodiment 19 is the method of any one of embodiments 12-24, wherein at least one of the homology arms is at least 1000 bp.

Embodiment 20 is the method of any one of embodiments 12-24, wherein at least one of the homology arms is at least 2000 bp.

Embodiment 21 is the method of any one of embodiments 12-20 and 22-24, wherein the artificial competence is induced by incubation in a solution containing divalent cations, by electroporation, or by ultrasound.

Embodiment 22 is the method of any one of embodiments 12-24, further comprising contacting the Spirulina with a pH balancer.

Embodiment 23 is the method according to any one of embodiments 22 or 24, wherein contacting the Spirulina with the pH balancer is prior to contacting the Spirulina with the vector.

Embodiment 24 is the method according to any one of embodiments 22 or 23, wherein a pH of a media containing the Spirulina and the pH balancer is about 7 to about 8.

Embodiment 25 is a Spirulina lacking at least one protein as a result of introducing a modification at the loci of the protein by transformation of the Spirulina with at least one DNA construct comprising a sequence homologous with at least a portion of the loci and the modification and integration of the DNA construct at the loci, the Spirulina being otherwise capable of functioning in its native manner.

Embodiment 26 is a Spirulina comprising an exogenous gene encoding phycocyanin as a result of introducing the exogenous gene at a targeted loci by transformation of the Spirulina with at least one DNA construct comprising a sequence homologous with at least a portion of the targeted loci and the exogenous gene and integration of the DNA construct at the locus, the Spirulina producing an increased amount of phycocyanin relative to wild-type Spirulina and maintaining the transformation throughout multiple generations.

1. A method for preparing a composition comprising a non-living, non-naturally occurring Spirulina, wherein the method comprises:
inducing competence in the Spirulina;
contacting the Spirulina with at least one vector comprising an exogenous nucleotide sequence, wherein the exogenous nucleotide sequence is then stably integrated into the Spirulina genome;
growing the Spirulina for at least five generations, wherein the exogenous nucleotide sequence is stably integrated into the Spirulina genome during each generation;
after the at least five generations, collecting a biomass of the Spirulina, and drying the biomass.

2. The method of claim 1, wherein the Spirulina is contacted with an osmotic stabilizer.

3. The method of claim 1, wherein the Spirulina is Spirulina platensis or Arthrospira maxima.

4. The method of claim 3, wherein the Spirulina is Arthrospira platensis NIES-39 or Arthrospira sp. PCC 8005.

5. The method of claim 2, wherein the osmotic stabilizer is at least one of polyethylene glycol, ethylene glycol, glycerol, glucose, or sucrose.

6. The method of claim 1, wherein the at least one vector is a DNA vector, a linear vector, a circular vector, a single stranded polynucleotide, or a double stranded polynucleotide.

7. (canceled)

8. The method of claim 1, wherein competence is induced artificially.

9. The method of claim 8, wherein artificial competence is induced by electroporation, incubation in one or more chemical solutions, or heat shock.

10. The method of claim 1, further comprising contacting the Spirulina with a pH balancer.

11. The method according to claim 10, wherein contacting the Spirulina with the pH balancer is prior to contacting the Spirulina with the vector.

12. The method according to claim 1110, wherein a pH of a media containing the Spirulina and the pH balancer is about 7 to about 8.

13. A non-living, non-naturally occurring dried biomass of Spirulina, the Spirulina comprising an exogenous nucleotide sequence,
wherein the exogenous nucleotide sequence is stably integrated into the Spirulina genome.

14. The Spirulina of claim 13, wherein the Spirulina is Arthrospira platensis or Arthrospira maxima.

15. The Spirulina of claim 14, wherein the Spirulina is Arthrospira platensis NIES-39 or Arthrospira sp. PCC 8005.

16. The Spirulina of claim 13, wherein the exogenous nucleotide sequence is stably integrated into the Spirulina genome for at least 10 generations.

17. The Spirulina of claim 16, wherein the exogenous nucleotide sequence is stably integrated into the Spirulina genome for at least 50 generations.

18-21. (canceled)


SEQUENCE LISTING (27 sequences)

SEQ ID NO: 1 - DNA, 6994 bp, synthetic construct
  misc_feature 1..6994: note = pApl-pilA plasmid backbone including addA gene with homology arms and restriction sites for Asd (GGCGCGCC) and EcoRI (GAATTC)
  source 1..6994: mol_type = other DNA
gaattcacatacgcggccgcctgggccttgagctcgaatttcctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccaagcttgcatgcctgcaggtggcgcgccgaatgacgctaacagcaggacctatattgggaattaggttgaggagtccggcgagaatggcattggcgaaaattaaggggactctcagtatccacaagccgactactgttagtagggtgacaccaatcatgttgagtaaggcaccgattagccaacgacctagggctatttcgcaagtatcgagaatgtcgttcattcgccttcggtaaaaggcgggaattagttgtaagctgctgcgacggtaggcttgggggtcggctaaaagcatgagggtgaggacgaggattaataagatgctgactaatccttctactgtggttccgacgacgaccccaaaaccgccgattaattgattgctgagggcttgggcttgttggataatttgttcgatgcggatctgtctcaggtcgcgactgacttctgatagatattcccgtaagggtccgttgaggtcggtgcgtaaatagtcaattaacaggttcagcagctttagaccttgttcgagaccggcggggacctgtttggctagttgttctaattggttgattaggggggggactaccaataaaaagccgatggtaatccctaagaagaatagactaatggataggaagacggcaagcgatcgcggtagtttcagacgctggaattgacgggtgagacggtttaaggcggtggctaggacgacggcggcgaaaatcatcagcagggcttgtttaatccgccacaggatgtatatagcgattaagaaagctgtgaagcctatccatgtacctagggtcattgcgactgttgagggtgagagaatttgcttttctacaggcgatcgcctgtacaatagttatctatcatagaatactagattctgtggtgggattccaccaaagccagccagctacctatgacgcaacccagatatgatgaacaggaaaatatatctcgccacgcagcggtagatgaaggatgctgcgaggatatggaacgaaaatatgggtggaatcttgtgcgaattgaggtaacctcagatcctatccttgaggttgattgcgtatttctggggaaaacggagtttcctcggccctataatgaaactgataaggaggaagattgatgcgaaaatacatcatttttcgggctgataagggtgaacctggttggaaagaacgtaaactccaacatacccagggtttaactagaattttggctgaacatttcgactcttctgatcaacctattcctgagttgggatatcgaccgagggagtttattcgtattgatgggttgcataatcccactgaacatggctacagcacccattatcgtcagggtgactgggaagtaactagggtagaaagttactctcctgatgtgtctatggctgagtttgatatgatcgtgatttgcttttgcaaatatagccctgttaatggtcctgtgcaacctatgccagagcgtcgagtgtctgtagatgggttcgctggggatgagaagcggttggagcaatggcacgagtcccaaaagaaaccgactgaggtttagttggtccccggtttgcttatttgactagccagattcgccacgacaacagaaccgccagcagggaagaggggaatagaaccgcgaaaagagcgatcgcattggtttcgggaatggcgaaatttggcccgccatatttgatcagtaaggatagccctagggataggatcaggattttggccaccaataacagtttgttgtccatttatatataggatgcgatgggatgcgatcgcctatattttaccaattggtggcggccttaaccccctaagccctatgtctgacacccagtcaccccaaccgccttagtcggtattccccactgggtgggcgatacacgatttcccggaaaaattgaaaaaaaattttgtggcaggtgatatgatttgcgatcgaggtgggtaagttagaactacagaaaacaaataacctcatttcaccccgaggagaacgaattatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataacagataatctagcagtttaggtttcagatggagtcagctagggtctttaaccccatcgtcaactataagatggagagatggcatacggtgacactgttaaagccataactatattgctcttgttgataaaatcagaaactgaggccacttgagagtattgtaaataggtagtacagtagtgctacctattttgttttaaaataactaattgagtttatcgctgggactatggttgataatcagatgtcggaaaagcagaatattttatcagcagaagctgatatttacttgcataaaagggattatcaacaagcagccattttatatgaacaagccatcaatctcaatcccgactataaatataattactggaaactggggttaaccctgctgctacaggagcgggaggaagaagcccagaccacctggctggtggcgatgagtgatggagacccagaacagattgaccagtggacgatagacttactcgaggtgctatcatcagaagccgatgcacgaacctccgccgaagactattccgtggcttgggggattcgccaacacattttagaaataaacccccatggtattgataatatattaaagttaattgatttgtcaattaccctgacaacctataccggaaatgagatcgcagaatatggactggttcccttactgcaacaggagtccatcaatgtcaatccagatttactactgcacctgtggaaaaaactcctagttgtagatgcaattaatcccctatctctggaattgggagaggcggtgattaaccatcttggtgagcaaccggagtttgtcactagcttaatccctaaattatacgatattgcctattcctttggtgaaataggtatagccaagggctatggagagttgttgttgaacctggcaccgcatcatcaagagttgctgagggctctggctcaattttgtattcaattaaccgagtatgaccatggcattgaatatgccagacgttcctatcatctcactgaatccccagatcagcaagccctggatagcaattttatgatcagtggtctgatgtctcaagggggatgcacaaagcaagtgcgagaggtggtcaaccatcaggaatcattgctggatgctatgctgaaagacccgcccctagacttgggaactggggttatgcaattatataataccagctttttctttccttatgttagagattcacctgctgtcaactcacaaataaaagcccaagttgctcaaatttgccaagccaacttagaaacggctcaggcagaaaatatcgccacatatcaagcccggaatttacagaaacgccatcaatccgcgaccaatcaacccctaaaaattggttatatatcttattgtttccggcggcattctgtgggttggatatctcgctggttatttcaacatcacgatcgcgataaatttgaaatttatgcctatatgattggggcagaaaatcgacaagatgctctccaaaactggtatgcagaacaagccaacaagtcttatcaatatggaatagtaagcacagaagttgccgagcaaatttctgaagaccagattgatattctcattgatttagatagcctgacattaaccaatacctgcgccattatggcacttaaacccgcacctgtacaagtcacctggttgggatgggatgcctctgctatacccaccgttgactattttattgctgacccctatgttttgccagaaaatgcccaggattattactcagaaaccatctggcgactacctcaaacctatgtggccgtcggtggcttcgaggtcggcattcccagtttgcgaagagaggatatcaatattcccgaaaatgccattgtttattttaccgcccaaagggggccgaaatacaacccagaactagcccaattgcaaatgcaaattctgaaacaagtacccaatagttatttggtaattaagggattcgataaagagcaatc

SEQ ID NO: 2 - DNA, 5967 bp, synthetic construct
  misc_feature 1..5967: note = pApl-pilA8005/aadA plasmid including aadA gene flanked by homology arms
  source 1..5967: mol_type = other DNA
gcctgggccttgagctcgaatttcctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccaagcttgcatgcctgcaggtggcgcgaacagaagacgaccaacactttctccctgatgggttgcgccctgatcaatgtcctttaaaccctgatacgttcttcaaccgtcgaacctagtccaaccgagtagcccataactgaccaccctatttaatcatgttgcattctgagcggaaccatgttcaaccccaaactatcccccaagttctcccggtccagtgaatctggctacaccctgcttgagtccatcatggctatggtggtggtcagcatcctgatggtcggcatttctccggtgttgggttttgccttcgccacacgggtacaggctcggcgcatagaactagccagcagagccgctaacagttatatcagtgctgtccgtactgacccaaccaataccaacttggtgctgttcaatcccagtgaccggaacgctggaccaactgcaactggtagcttgaactgtggggctggtcgagcctgtaccaccccagccaacttgttctgtgtggataatgacggaagtgggggatgtgaaaccgatagccccacggatatgattgttcagggaattggcataaatgagtgtagcgacgagctagagaatggttatcagttgattgtcagggtgtatcgtgctgacgcatttgcccaatcgggacaactagagggggctaatgatgacgggtctagtaatttagcgagttcccgagtcggtttaggaaaccccagagaccccctagtgcaggtgcgaaccgagattagccccactagcaccagttcctttgggagtttacgtgaccgtttagcaggatgcagttctgacgatgaaggggattaataggtttggtgatggttcccatggagtgcaaaagtaagattatcccggtagagaggtagttatgctgtccctagttcggttttggttaatttgtgtattgaaatctaggtcaaaaaccacccaggggaatgacttaggcttcacgatgattgagttattgatttcggctgttgtggctagtatgatcatcacgcccatcctattctttgtggtggatgttttacgaaccgatacccgggaagaaatcacagccagatcggaacaggagttacaggcggcagcagatttcattcgtcgcgacctctcccaggctatctatatctacgacggtgaaggggttaatgcgattaggagccaacttcctgattccgataatgtggtattggtgttctggaaacaggagtttcgcgaaggtgtgattcctcacggtgacgtccccaggccagttgctgctaatcattgcgatacgaggcgttgcgatgatgtccaagttttatctttagttgcttactatttacaaacagaaagagagcctaatagtccttggtctgatcaggcgcgtatttctcgcttacaaatcaacgatggtgtcatcgatccgaatccagaagatgaatgcacccaaccaccatgttatgtcaaactgggtgataaggaattcaaaccagaccgaggttttgatcgacctcctggccgtacaagcatgaatgcttgggaagcaaaagagggcgagcagattgaagcatcacgagaagttctgattgattacatagacaaaacagaactaacaggtgatatagattgtgaatctcgtggcatcagtaatgatgatagtgaggatttactatccgtgggtaagaattctgaggggaatcctatctatggcttttttgtatgtgtagactcccgtaatacagcagctttagttaacctgagaatgaatgcggcaattcgcattgatccgaatgctacagaattcgacgaggataggcgatcgctttttccagagaccagctttttggttaaaggcttaggtgttttcaattaaccgataaaatcagggaatttataataaaaggaacctataaaatgagtattttaattaacccaaaatcggtgaatcccaggcgcaccaaaaaccagtcgggcttcaccatcattgagacgttggtggtagttttaatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataagtagttataactagccaattgtaacacaaaaaattaattatctactgaatattttacaaaactttacccccaatttcggaaaacatagtttagaatacaccaccaaagatatcacccgtaacgaggccttttacgagtgagattagttgtgtatgctgcggttagctgacctacacgacatctatccttgtgacccaggttaaataatggaaaatacattaattaagcacttgcttctggttcagaaacaggctattaatttagtgcctgaaaaacctgttagcagtgactatcagggtgcgaaatcgcgaaaatcctctacagaatcagggttttctttgttagaagtgttaggggtgatggtgattatatcaatactaggtgggatagcggttccgacttggttaggtttcgcgaataaccagaaactgcaaacctctatccggcgactcaactgggctatccactcagccaaaagtgaggctatactatctagtacatcatggcaagcgagtatcagggaatatgggggacgagtacaggtagcagtccatcccgcgaagactccccctggtcaactcccagaaggttcatggactaacctagaaccggggatagaaattgacaaccgggaaaggaacaccaggaataaatatgaaactaccctgcgaagagtagacccggacaccaaccaagtccgaaacgcggggacggtgtaccgagttttattcaactaccaaggctgtccggtctataacccgtctaatttatgcacgcaaacttctctagctgcgaagggacgcattgctatccatcatcctgaagttccaggtcaaaagcgatgtatcatcatttcgacgctgataggtgcgactcgcattggtcagcaacagcaaagaccggatcaaaatggctactattgccattaattaataattaataattaataattaataattattaattgggtagcgaccctgtgggcc

SEQ ID NO: 3 - DNA, 8117 bp, synthetic construct
  misc_feature 1..8117: note = pApl-NS1/Prs-crtW-crtZ plasmid including aadA gene, crtW, crtZ, and homology arms
  source 1..8117: mol_type = other DNA
gcctgggccttgagctcgaatttcctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccaagcttgcatgcctgcaggtggcgcgccaggtccgaagaagaacgcaccatagctgctggtaaagaagcccgcgccagggctgaacaactcctgcaaactgtccgcgctgagttggaaaggttgcgactcgaagctgataccgtcctacctgcagaggcggagcgagaagccagagaactgcaagccagaggtaatgcggccgctttagcggaaaattcccgcgcggcggcggaagtaaacgacctgattaacgcagtttggcaggaaatgggcgacgacgcatcggaactgttttacttacaacaaattgagatggtcttgcgcgaagctgcccaaattccccgccgggttagtctggaatatgtcaatgttattgatagtggagacggggaagcgatcgctggtttggttcgcgcctatcccgaaattttccgccaattcctcgaaagtgtcgatcgcactttgggtattaatgtatctaggactttgggggaacataacaagcagaaattaaataacaattagtcaatttgatggcaaccaaacacaggtacaggaattatggaaattatcatcgccttattagcagccctgggtttaggaaccggagcgactgtatttattatccgcaatctctactatatttgtcagcctagcgaagtgttgatttttgccggaggtcgtcgccctacacaagatgatcgcactgtgggatatcgcctggtgaaaggtggtagcagcattcggacacccctgctagaacaagcctttcacatggatttaaccaatatgattattgagttaaaggtggctaatgcttactcgaaaggaggcattcctttacaggttgatggggtggcgaatattaaaattgctggggaagaacccacaattcataatgcgatcgagcgtttgttggggaaaactcgccaggaaattgaacgcattgctaaagataccctggaaggaaacctgcgcggggtattagccagccttactcctgaacaggttaatggggataaattggcttttgctaaaagtctcctcgaagaagctgaggatgacttagaacaactggggctgattttagatactctccaaattcaaaatatttctgatgacgtgggatatcttgactccatcggacgacaacaacgggcggaattgtttagagattctcgtgtcgctgaagcacaagctaaggctgatgctgctatccgtactgcggaaaatcataaaatgactcagttgaaaaagttggaaactgaggtagaggtatctaaggcggaagctgaacgccgggtggctgatgctatgactaaacgggcggcggtggtagcagagtcggagtcggaaacagcggctgaggtggctaggactcaggctgaggtctctgttcaaaaagaaaggattaagcaggtagaacagcaactacaagctgatgtggtcgcaccagcagaggcggaatgtaaaaaggcgatcgctagggcgcggggggatgctgcccaaattattgaagatgggaaagcacaagctgaagggacacgacgactggctgagtcttggaaagcagccggtcggaccgtcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcatacgctcacaattggtaccggtgataccagcatcgtcttgatgcccttggcagcaccctgctaaggaggcaacaagatgacggctgcggtcgctgagccgcgaatcgtgcctcgtcaaacctggatcgggctcacacttgcagggatgattgtggcaggctgggggagtcttcacgtctatggagtctactttcatcgctgggggacaagtagcctcgttattgtgccagcaatcgtggctgtccagacctggctcagtgtgggcctattcattgtcgcacacgacgctatgcacgggtcgctggcgccgggtcggcctcgcctaaacgcagccgtaggcagactcaccttagggctctatgccggtttccgatttgatcgccttaaaacggcccaccacgcacatcacgccgcccccggtactgccgatgaccctgatttctacgccccagcaccgcgagcgtttttgccttggttcctcaacttctttcgcacttactttggctggcgtgagatggctgttctgacagctttagtcctaatagccttgtttggtttgggtgcccggccggcaaatcttctcacgttctgggcagctccggcgctgttaagtgctctacagctattcacttttggcacgtggctgccgcaccgccacactgatcagccttttgcggacgcccaccatgctcggtcttccggttacggcccggtgctcagcctgcttacctgttttcactttggtcggcaccacgaacaccatctgacaccgtggcgcccctggtggcggctatggcgaggtgagagctaaacacaggaaacaggaattagatatgctgtggatttggaacgcgctgatcgtgtttgtcacagtcatcgggatggaagttattgccgccttggcacacaaatacatcatgcacggctggggctggggatggcatctgtctcaccacgaaccccgcaaaggcgctttcgaagtcaatgatctgtatgctgtcgtgtttgcagccctgagcattttgctcatctacctgggctcgaccggtatgtggccgttgcaatggattggtgccggcatgaccgcctacggtctgctgtatttcatggtgcacgatggcctggtccatcaacggtggccttttcggtatattccccgaaagggctacctgaagcgcctatatatggcccatcgaatgcatcacgcagtccgggggaaggaagggtgtgtgagttttgggtttctatatgctcccccgttgtcgaaactgcaagcaaccctgcgtgagcgtcatggcgctcgcgctggggcggcgcgggacgcgcaagggggcgaagatgaaccggcatcggggaaataaactagtcatcgagctagcaagcttggccggatccggccggatccggagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgagtcaccccaaccgccttagtcggtattccccactgggtgggcgatacacgatttcccggaaaaattgaaaaaaaattttgtggcaggtgatatgatttgcgatcgaggtgggtaagttagaactacagaaaacaaataacctcatttcaccccgaggagaacgaattatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataactagctggcctggaagggtgggaaagtgttaacaactggttgacaatattccgcttttccaggtcttgtcgaagtacgactacgggtattgatgtggcgacggctgttcaaaattggggcggcggttcttctcaacctcaacctacttccacagctaaaagtctcaaatcttcccgcactaatggtcaagctgttcgccgcactaatggcgcagcacctaagccagaagatactacacaaagcctacagagcgaggttaaacaactacttgatcatgtcgcacaaaatactaagaataatgctgaggctgaagaggcgatcgcccaggctttgcgacagtatcctaacctcaagcgtcgtttacagatgctttttaagtctcagggaactcaggcgatccgaaatatgtttaatcaccctactatctacatccctaccgctatgctgcaaatgtggctagaacaggattagagcgtattcccccccctctcgtctagggtgggggatctgtctctgtaactgtatatgcacaaaatggcgtttttgtcaataagccgaaaaatggaccccgcttttaaccccaaaactcgtgcatcgccagttttattttttggtaagataataattataaccgcgacaaaataagggttttagcctttatttttggaaatagacacagtaagcccttgggtgcgatttccttgagcataccataacccccataatctcgtcaactttttgcgaggtatttgccataaaattgtattaattcttaacaatattttaaaaataaaatctatctagccgtataaattagtaaaatttatgtaatttcccgattgagggggtggggtgtgggggggtttatagtcaaattaatgtgattggatctcatttaacctctgacgtactacttgcatatcctgccacattaaccatttaggtttaccaacatctcgcgaggaatttctgagtaggtatgcagggtgaaaaatcggcatacattgacgaccttcccactgaaaccactgaccgcgaattttagtaatcgggcgtttatctcctagaatagacttaactgcagtcgccccagttaggaggataatttgaggattaatgaggcggatttgttctaatagataaggcttacaggcttggagttctgaagcggttggggtgcgattttccgggggacgacagcggatagcattagaaatataaacttcggtatctgtactcagtcctacggagtctaaaatattatcaagaagttttccagatctaccgacaaacggaagaccttgttcatcttcatgttgtcctggtgcttctccgataatcataatgggggcggtggggttccctcgaccaatcacagcatgagtgcggttttttccaagttcacagcgcgagcacctattacaatgatcggcaatttgtgcgagagtttgataggttcccggcggtatgggagtcttggcattttggggaatttcttctaggtcaaaatctaccgacttaggggattgtaatgggtcttgattaacactaagaatatcgaacaggctgagttgttgggaattcacatacgcggcc

SEQ ID NO: 4 - DNA, 8146 bp, synthetic construct
  misc_feature 1..8146: note = pApl-NS1/aadA-cpcBA plasmid backbone including aadA, cpcA, and cpcB genes with a p15A origin of replication
  source 1..8146: mol_type = other DNA
aggtccgaagaagaacgcaccatagctgctggtaaagaagcccgcgccagggctgaacaactcctgcaaactgtccgcgctgagttggaaaggttgcgactcgaagctgataccgtcctacctgcagaggcggagcgagaagccagagaactgcaagccagaggtaatgcggccgctttagcggaaaattcccgcgcggcggcggaagtaaacgacctgattaacgcagtttggcaggaaatgggcgacgacgcatcggaactgttttacttacaacaaattgagatggtcttgcgcgaagctgcccaaattccccgccgggttagtctggaatatgtcaatgttattgatagtggagacggggaagcgatcgctggtttggttcgcgcctatcccgaaattttccgccaattcctcgaaagtgtcgatcgcactttgggtattaatgtatctaggactttgggggaacataacaagcagaaattaaataacaattagtcaatttgatggcaaccaaacacaggtacaggaattatggaaattatcatcgccttattagcagccctgggtttaggaaccggagcgactgtatttattatccgcaatctctactatatttgtcagcctagcgaagtgttgatttttgccggaggtcgtcgccctacacaagatgatcgcactgtgggatatcgcctggtgaaaggtggtagcagcattcggacacccctgctagaacaagcctttcacatggatttaaccaatatgattattgagttaaaggtggctaatgcttactcgaaaggaggcattcctttacaggttgatggggtggcgaatattaaaattgctggggaagaacccacaattcataatgcgatcgagcgtttgttggggaaaactcgccaggaaattgaacgcattgctaaagataccctggaaggaaacctgcgcggggtattagccagccttactcctgaacaggttaatggggataaattggcttttgctaaaagtctcctcgaagaagctgaggatgacttagaacaactggggctgattttagatactctccaaattcaaaatatttctgatgacgtgggatatcttgactccatcggacgacaacaacgggcggaattgtttagagattctcgtgtcgctgaagcacaagctaaggctgatgctgctatccgtactgcggaaaatcataaaatgactcagttgaaaaagttggaaactgaggtagaggtatctaaggcggaagctgaacgccgggtggctgatgctatgactaaacgggcggcggtggtagcagagtcggagtcggaaacagcggctgaggtggctaggactcaggctgaggtctctgttcaaaaagaaaggattaagcaggtagaacagcaactacaagctgatgtggtcgcaccagcagaggcggaatgtaaaaaggcgatcgctagggcgcggggggatgctgcccaaattattgaagatgggaaagcacaagctgaagggacacgacgactggctgagtcttggaaagcagccggtagtcaccccaaccgccttagtcggtattccccactgggtgggcgatacacgatttcccggaaaaattgaaaaaaaattttgtggcaggtgatatgatttgcgatcgaggtgggtaagttagaactacagaaaacaaataacctcatttcaccccgaggagaacgaattatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataactagctggcctggaagggtgggaaagtgttaacaactggttgacaatattccgcttttccaggtcttgtcgaagtacgcggaccgttgaaggggtgagaaggaaggcgaatgttgatttatgaagtttgattaacatttgtatcaaaatataaaattcttctcataaaccctgtacaatcttttaagatttcggaaagtgttctaggatactgaagaaatgaaccacggggcaattgttaaaagcctttgtcgatggttcgccccggaaggggtcttaggaggtgacaccgatggattgattgtcgtgatcattcatggtgtgtccaatcccaactcaactctaagcaagtcaacaagtaggagataaatccatgtttgatgccttcaccaaggtggtttctcaagctgatactcgcggcgaaatgctgagtacagctcaaatcgatgctctgagccaaatggttgctgaaagcaacaaacgtttggattctgttaaccgcattaccagcaacgcttccaccattgtttccaacgctgctcgttctttgttcgcagagcaaccccaactgattgctcccggtggaaacgcctacaccagccgtcgtatggctgcttgcttgcgtgacatggaaatcatcctgcgctatgtaacctacgctgtgtttgctggcgatgcaagtgttctcgaagatcgttgcttgaacggtttgcgtgaaacttacctggctttgggaactcccggttcttccgttgctgtcggtgttggcaaaatgaaagaagctgctctggcgatcgttaacgatcccgcaggtatcactcctggtgattgtagcgctttggcttcagaaatcgctggttactttgaccgtgctgctgcagcagtttcctaatcaagcagatccatagcatataacaattgaaacagtttagctgaagtctaagtgactggacttctgtttgttacctaattttttgtaaaccaatcgggagataactcgagaatgaaaacccccctaaccgaagcagtttctatcgctgattcccaaggtcgtttcctaagcagcaccgaaatccaagtagcttttggccgttttcgtcaagccaaagctggtctggaagctgctaaagctttgacctctaaagctgatagtctgatcagtggtgctgcccaagcagtgtacaacaagttcccctacaccacccaaatgcagggacctaactacgcggcagaccaacgcggtaaggacaaatgtgctcgtgacataggctactacctgcggatggtaacttattgcctgattgctggtggaactggccccatggatgagtacctgattgccggtattgatgaaatcaaccggactttcgagctttctccaagctggtacattgaagccctgaaatacatcaaagctaaccacggtttgtctggtgacgctgctgttgaagctaactcctacctcgactacgcgatcaacgccctgagctagactagtcatcgagctagcaagcttggccggatccggccggatccggagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgactacgggtattgatgtggcgacggctgttcaaaattggggcggcggttcttctcaacctcaacctacttccacagctaaaagtctcaaatcttcccgcactaatggtcaagctgttcgccgcactaatggcgcagcacctaagccagaagatactacacaaagcctacagagcgaggttaaacaactacttgatcatgtcgcacaaaatactaagaataatgctgaggctgaagaggcgatcgcccaggctttgcgacagtatcctaacctcaagcgtcgtttacagatgctttttaagtctcagggaactcaggcgatccgaaatatgtttaatcaccctactatctacatccctaccgctatgctgcaaatgtggctagaacaggattagagcgtattcccccccctctcgtctagggtgggggatctgtctctgtaactgtatatgcacaaaatggcgtttttgtcaataagccgaaaaatggaccccgcttttaaccccaaaactcgtgcatcgccagttttattttttggtaagataataattataaccgcgacaaaataagggttttagcctttatttttggaaatagacacagtaagcccttgggtgcgatttccttgagcataccataacccccataatctcgtcaactttttgcgaggtatttgccataaaattgtattaattcttaacaatattttaaaaataaaatctatctagccgtataaattagtaaaatttatgtaatttcccgattgagggggtggggtgtgggggggtttatagtcaaattaatgtgattggatctcatttaacctctgacgtactacttgcatatcctgccacattaaccatttaggtttaccaacatctcgcgaggaatttctgagtaggtatgcagggtgaaaaatcggcatacattgacgaccttcccactgaaaccactgaccgcgaattttagtaatcgggcgtttatctcctagaatagacttaactgcagtcgccccagttaggaggataatttgaggattaatgaggcggatttgttctaatagataaggcttacaggcttggagttctgaagcggttggggtgcgattttccgggggacgacagcggatagcattagaaatataaacttcggtatctgtactcagtcctacggagtctaaaatattatcaagaagttttccagatctaccgacaaacggaagaccttgttcatcttcatgttgtcctggtgcttctccgataatcataatgggggcggtggggttccctcgaccaatcacagcatgagtgcggttttttccaagttcacagcgcgagcacctattacaatgatcggcaatttgtgcgagagtttgataggttcccggcggtatgggagtcttggcattttggggaatttcttctaggtcaaaatctaccgacttaggggattgtaatgggtcttgattaacactaagaatatcgaacaggctgagttgttgggaattcacatacgcggccgcctgggccttgagctcgaatttcctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaatagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccattcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccaagcttgcatgcctgcaggtggcgcgcc

SEQ ID NO: 5 - protein, 458 aa, Acinetobacter baylyi
MRPLHPIDFIFLSLEKRQQPMHVGGLFLFQIPDNAPDTFIQDLVNDIRISKSIPVPPFNNKLNGLFWDEDEEFDLDHHFRHIALPHPGRIRELLIYISQEHSTLLDRAKPLWTCNIIEGIEGNRFAMYFKIHHAMVDGVAGMRLIEKSLSHDVTEKSIVPPWCVEGKRAKRLREPKTGKIKKIMSGIKSQLQATPTVIQELSQTVFKDIGRNPDHVSSFQAPCSILNQRVSSSRRFAAQSFDLDRFRNIAKSLNVTINDVVLAVCSGALRAYLMSHNSLPSKPLIAMVPASIRNDDSDVSNRITMILANLATHKDDPLQRLEIIRRSVQNSKQRFKRMTSDQILNYSAVVYGPAGLNIISGMMPKRQAFNLVISNVPGPREPLYWNGAKLDALYPASIVLDGQALNITMTSYLDKLEVGLIACRNALPRMQNLLTHLEEEIQLFEGVIAKQEDIKTAN

SEQ ID NO: 6 - protein, 446 aa, Streptomyces coelicolor
MTPDPLAPLDLAFWNIESAEHPMHLGALGVFEADSPTAGALAADLLAARAPAVPGLRMRIRDTWQPPMALRRPFAFGGATREPDPRFDPLDHVRLHAPATDFHARAGRLMERPLERGRPPWEAHVLPGADGGSFAVLFKFHHALADGLRALTLAAGVLDPMDLPAPRPRPEQPPRGLLPDVRALPDRLRGALSDAGRALDIGAAAALSTLDVRSSPALTAASSGTRRTAGVSVDLDDVHHVRKTTGGTVNDVLIAVVAGALRRWLDERGDGSEGVAPRALIPVSRRRPRSAHPQGNRLSGYLMRLPVGDPDPLARLGTVRAAMDRNKDAGPGRGAGAVALLADHVPALGHRLGGPLVSGAARLWFDLLVTSVPLPSLGLRLGGHPLTEVYPLAPLARGHSLAVAVSTYRGRVHYGLLADAKAVPDLDRLAVAVAEEVETLLTACRP

SEQ ID NO: 7 - protein, 457 aa, Alcanivorax borkumensis
MKALSPVDQLFLWLEKRQQPMHVGGLQLFSFPEGAGPKYVSELAQQMRDYCHPVAPFNQRLTRRLGQYYWTRDKQFDIDHHFRHEALPKPGRIRELLSLVSAEHSNLLDRERPMWEAHLIEGIRGRQFALYYKIHHSVMDGISAMRIASKTLSTDPSEREMAPAWAFNTKKRSRSLPSNPVDMASSMARLTASISKQAATVPGLAREVYKVTQKAKKDENYVSIFQAPDTILNNTITGSRRFAAQSFPLPRLKVIAKAYNCTINTVVLSMCGHALREYLISQHALPDEPLIAMVPMSLRQDDSTGGNQIGMILANLGTHICDPANRLRVIHDSVEEAKSRFSQMSPEEILNFTALTMAPTGLNLLTGLAPKWRAFNVVISNIPGPKEPLYWNGAQLQGVYPVSIALDRIALNITLTSYVDQMEFGLIACRRTLPSMQRLLDYLEQSIRELEIGAGIK

SEQ ID NO: 8 - DNA, 2595 bp, synthetic construct
  misc_feature 1..2595: note = Codon-optimized S. cerevisiae phosphatidate phosphatase (PAH1)
  source 1..2595: mol_type = other DNA
atggaattccaatatgttggtcgggctttgggtagtgttagtaaaacgtggtcgagtatcaaccccgccaccctgagcggcgctatcgatgtcattgtcgtggaacaccccgatggccggctcagttgtagccccttccatgtgcgctttggtaaattccagattctgaaacccagccaaaagaaagtccaggtctttattaacgagaaactgtcgaatatgcccatgaaactctcggatagcggcgaggcgtacttcgtttttgagatgggtgatcaagtgacggatgtcccggatgaactgctcgtctcgccggtcatgagtgccacgagtagtccgccccaatcgccggaaacctcgattctcgaaggcggtaccgaaggcgagggcgaaggtgagaatgaaaataagaaaaaggaaaagaaggtgttggaggagcccgactttctggacattaatgacaccggtgacagcggcagcaagaacagtgagacgacgggttcgctctcgccgaccgaaagtagtacgacgacgccgcccgatagcgtcgaggaacgcaagttggtcgaacaacggaccaagaattttcagcaaaagctgaataagaaactgaccgaaatccatattccgagcaaattggacaataacggtgatttgctcctggacaccgagggttataagccgaataaaaacatgatgcacgacacggatattcagctgaagcaattgctcaaggatgagttcggtaacgatagcgatatttcgagcttcatcaaagaagacaagaatggcaacattaaaatcgtgaacccctatgagcatttgaccgatttgagtcccccgggtacgcccccgaccatggccacgagtggcagtgtcctgggcttggatgcgatggagagtggttcgacgctgaacagcttgagcagcagcccgagcggcagtgacaccgaggatgagacgagctttagcaaggaacagtcgtcgaagagtgaaaaaacgtcgaagaaaggcaccgcgggttcgggtgaaacggagaaacgctacatccgcacgatccggctcacgaatgatcagctgaaatgcctcaacttgacgtacggtgaaaatgacttgaaatttagtgttgaccatggcaaagccattgtgaccagcaaattgtttgtctggcgctgggacgtccccatcgttatcagcgacattgacggtacgattacgaaaagtgatgcgctgggccacgtcctcgccatgatcggcaaagattggacccatctcggcgtcgctaagctgttcagtgagatctcgcgcaacggttacaatatcctgtacctgaccgcgcgctcggccggtcaggctgacagtacccgctcgtatctccgcagtattgagcagaacggtagcaagctcccgaacggccccgtcattctgagccccgatcggaccatggctgccctgcgccgggaggtgattctgaaaaagcccgaagtctttaaaatcgcttgcttgaacgatatccgctcgctctatttcgaagactcggataacgaagtggacacggaggaaaagagcacgccgtttttcgcgggctttggcaatcggatcaccgatgcgctcagctatcggacggtcggcatcccgagtagccgcatcttcacgattaacacggaaggcgaggtgcacatggagctgctcgagctcgccggttaccggagtagctatatccatatcaacgaactggtcgatcacttcttcccgccggtgagcctggactcggtcgatctgcgcacgaacacgagcatggtcccgggcagcccgccgaaccgcaccctggataactttgatagcgaaatcaccagtggccgcaagacgttgtttcgcggtaatcaggaggaaaaattcacggacgtcaacttttggcgcgatccgttggtggacatcgacaacctctcggatatcagtaacgatgattcggacaatattgatgaagacaccgatgtgagccaacagtcgaacatcagccgcaaccgcgctaactcggtcaagacggccaaggtgaccaaggctccgcagcggaatgtgtcgggcagtacgaataacaatgaagttctggctgcgagtagtgatgttgaaaatgccagtgacttggttagcagccactcgagtagcggctcgacccccaacaagtcgacgatgagtaagggtgatatcggcaaacaaatctatctggaactgggctcgcccttggcgagtcccaaactccggtatctggacgatatggatgatgaggactcgaactataatcgcaccaagagccgccgggctagtagcgccgctgctaccagcatcgacaaggagtttaaaaagctcagtgtgagtaaagctggcgctcccacccgcatcgttagcaagatcaacgtgtcgaatgatgtgcacagtttgggcaacagtgataccgaaagccggcgggaacagagcgtcaatgaaaccggtcgcaatcagttgccgcacaatagtatggatgataaggatttggattcgcgggtgagtgacgagttcgatgacgatgagtttgatgaagatgagtttgaggattag

SEQ ID NO: 9 - DNA, 6936 bp, Triticum aestivum
  source 1..6936: mol_type = other DNA
atgggatccacacatttgcccattgtcggccttaatgcctcgacaacaccatcgctatccactattcgcccggtaaattcagccggtgctgcattccaaccatctgccccttctagaacctccaagaagaaaagtcgtcgtgttcagtcattaagggatggaggcgatggaggcgtgtcagaccctaaccagtctattcgccaaggtcttgccggcatcattgacctcccaaaggagggcacatcagctccggaagtggatatttcacatgggtccgaagaacccaggggctcctaccaaatgaatgggatactgaatgaagcacataatgggaggcatgcttcgctgtctaaggttgtcgaattttgtatggcattgggcggcaaaacaccaattcacagtgtattagttgcgaacaatggaagggcagcagctaagttcatgcggagtgtccgaacatgggctaatgaaacatttgggtcagagaaggcaattcagttgatagctatggctactccagaagacatgaggataaatgcagagcacattagaattgctgatcaatttgttgaagtacccggtggaacaaacaataacaactatgcaaatgtccaactcatagtggagatagcagtgagaaccggtgtttctgctgtttggcctggttggggccatgcatctgagaatcctgaacttccagatgcactaaatgcaaacggaattgtttttcttgggccaccatcatcatcaatgaacgcactaggtgacaaggttggttcagctctcattgctcaagcagcaggggttccgactcttccttggggtggatcacaggtggaaattccattagaagtttgtttggactcgatacctgcggagatgtataggaaagcttgtgttagtactacggaggaagcacttgcgagttgtcagatgattgggtatccagccatgattaaagcatcatggggtggtggtggtaaagggatccgaaaggttaataacgacgatgatgtcagagcactgtttaagcaagtgcaaggtgaagttcctggctccccaatatttatcatgagacttgcatctcagagtcgacatcttgaagttcagttgctttgtgatcaatatggcaatgtagctgcgcttcacagtcgtgactgcagtgtgcaacggcgacaccaaaagattattgaggaaggaccagttactgttgctcctcgcgagacagtgaaagagctagagcaagcagcaaggaggcttgctaaggctgtgggttatgttggtgctgctactgttgaatatctctacagcatggagactggtgaatactattttctggaacttaatccacggttgcaggttgagcatccagtcaccgagtggatagctgaagtaaacttgcctgcagctcaagttgcagttggaatgggtatacccctttggcaggttccagagatcagacgtttctatggaatggacaatggaggaggctatgacatttggagggaaacagcagctcttgctactccatttaacttcgatgaagtggattctcaatggccaaagggtcattgtgtagcagttaggataaccagtgaggatccagatgacggattcaagcctaccggtggaaaagtaaaggagatcagttttaaaagcaagccaaatgtttgggcctatttctctgttaagtccggtggaggcattcatgaatttgctgattctcagtttggacatgtttttgcatatggagtgtctagagcagcagcaataaccaacatgtctcttgcgctaaaagagattcaaattcgtggagaaattcattcaaatgttgattacacagttgatctcttgaatgcctcagacttcaaagaaaacaggattcatactggctggctggataacagaatagcaatgcgagtccaagctgagagacctccgtggtatatttcagtggttggaggagctctatataaaacaataacgagcaacacagacactgtttctgaatatgttagctatctcgtcaagggtcagattccaccgaagcatatatcccttgtccattcaactgtttctttgaatatagaggaaagcaaatatacaattgaaactataaggagcggacagggtagctacagattgcgaatgaatggatcagttattgaagcaaatgtccaaacattatgtgatggtggacttttaatgcagttggatggaaacagccatgtaatttatgctgaagaagaggccggtggtacacggcttctaattgatggaaagacatacttgttacagaatgatcacgatccttcaaggttattagctgagacaccctgcaaacttcttcgtttcttggttgccgatggtgctcatgttgaagctgatgtaccatatgcggaagttgaggttatgaagatgtgcatgcccctcttgtcacctgctgctggtgtcattaatgttttgttgtctgagggccagcctatgcaggctggtgatcttatagcaagacttgatcttgatgacccttctgctgtgaagagagctgagccatttaacggatctttcccagaaatgagccttcctattgctgcttctggccaagttcacaaaagatgtgccacaagcttgaatgctgctcggatggtccttgcaggatatgatcacccgatcaacaaagttgtacaagatctggtatcctgtctagatgctcctgagcttcctttcctacaatgggaagagcttatgtctgttttagcaactagacttccaaggcttcttaagagcgagttggagggtaaatacagtgaatataagttaaatgttggccatgggaagagcaaggatttcccttccaagatgctaagagagataatcgaggaaaatcttgcacatggttctgagaaggaaattgctacaaatgagaggcttgttgagcctcttatgagcctactgaagtcatatgagggtggcagagaaagccatgcacactttattgtgaagtcccttttcgaggactatctctcggttgaggaactattcagtgatggcattcagtctgatgtgattgaacgcctgcgccaacaacatagtaaagatctccagaaggttgtagacattgtgttgtctcaccagggtgtgagaaacaaaactaagctgatactaacactcatggagaaactggtctatccaaaccctgctgtctacaaggatcagttgactcgcttttcctccctcaatcacaaaagatattataagttggcccttaaagctagcgagcttcttgaacaaaccaagcttagtgagctccgcacaagcattgcaaggagcctttcagaacttgagatgtttactgaagaaaggacggccattagtgagatcatgggagatttagtgactgccccactgccagttgaagatgcactggtttctttgtttgattgtagtgatcaaactcttcagcagagggtgatcgagacgtacatatctcgattataccagcctcatcttgtcaaggatagtatccagctgaaatatcaggaatctggtgttattgctttatgggaattcgctgaagcgcattcagagaagagattgggtgctatggttattgtgaagtcgttagaatctgtatcagcagcaattggagctgcactaaagggtacatcacgctatgcaagctctgagggtaacataatgcatattgctttattgggtgctgataatcaaatgcatggaactgaagacagtggtgataacgatcaagctcaagtcaggatagacaaactttctgcgacactggaacaaaatactgtcacagctgatctccgtgctgctggtgtgaaggttattagttgcattgttcaaagggatggagcactcatgcctatgcgccataccttcctcttgtcggatgaaaagctttgttatggggaagagccggttctccggcatgtggagcctcctctttctgctcttcttgagttgggtaagttgaaagtgaaaggatacaatgaggtgaagtatacaccgtcacgtgatcgtcagtggaacatatacacacttagaaatacagagaaccccaaaatgttgcacagggtgtttttccgaactcttgtcaggcaacccggtgcttccaacaaattcacatcaggcaacatcagtgatgttgaagtgggaggagctgaggaatctctttcatttacatcgagcagcatattaagatcgctgatgactgctatagaagagttggagcttcacgcgattaggacaggtcactctcatatgtttttgtgcatattgaaagagcgaaagcttcttgatcttgttcccgtttcagggaacaaagttgtggatattggccaagatgaagctactgcatgcttgcttctgaaagaaatggctctacagatacatgaacttgtgggtgcaaggatgcatcatctttctgtatgccaatgggaggtgaaacttaagttggacagcgatgggcctgccagtggtacctggagagttgtaacaaccaatgttactagtcacacctgcactgtggatatctaccgtgaggttgaagatacagaatcacagaaactagtataccactctgctccatcgtcatctggtcctttgcatggcgttgcactgaatactccatatcagcctttgagtgttattgatctgaaacgttgctccgctagaaacaacagaactacatactgctatgattttccgttggcatttgaaactgcagtgcagaagtcatggtctaacatttctagtgacaataaccgatgttatgttaaagcaacggagctggtgtttgctcacaagaatgggtcatggggcactcctgtaattcctatggagcgtcctgctgggctcaatgacattggtatggtagcttggatcttggacatgtccactcctgaatatcccaatggcaggcagattgttgtcatcgcaaatgatattacttttagagctggatcgtttggtccaagggaagatgcattttttgaaactgttaccaacctagcttgtgagaggaggcttcctctcatctacttggcagcaaactctggtgctcggatcggcatagcagatgaagtaaaatcttgcttccgtgttggatggtctgatgatggcagccctgaacgtgggtttcaatatatttatctgactgaagaagaccatgctcgtattagcgcttctgttatagcgcacaagatgcagcttgataatggtgaaattaggtgggttattgattctgttgtagggaaggaggatgggctaggtgtggagaacatacatggaagtgctgctattgccagtgcctattctagggcctatgaggagacatttacgcttacatttgtgactggaaggactgttggaataggagcatatcttgctcgacttggcatacggtgcattcagcgtactgaccagcccattatcctaactgggttttctgccttgaacaagcttcttggccgggaagtgtacagctcccacatgcagttgggtggccccaaaattatggcgacaaacggtgttgtccatctgacagtttcagatgaccttgaaggtgtatctaatatattgaggtggctcagctatgttcctgccaacattggtggacctcttcctattacaaaatctttggacccacctgacagacccgttgcttacatccctgagaatacatgcgatcctcgtgctgccatcagtggcattgatgatagccaagggaaatggttggggggcatgttcgacaaagacagttttgtggagacatttgaaggatgggcgaagtcagttgttactggcagagcgaaactcggagggattccggtgggtgttatagctgtggagacacagactatgatgcagctcatccctgctgatccaggccagcttgattcccatgagcgatctgttcctcgtgctgggcaagtctggtttccagattcagctactaagacagcgcaggcaatgctggacttcaaccgtgaaggattacctctgttcatccttgctaactggagaggcttctctggtggacaaagagatctttttgaaggaatccttcaggctgggtcaacaattgttgagaaccttagggcatacaatcagcctgcctttgtatatatccccaaggctgcagagctacgtggaggggcttgggtcgtgattgatagcaagataaatccagatcgcattgagttctatgctgagaggactgcaaagggcaatgttctcgaacctcaagggttgatcgagatcaagttcaggtcagaggaactccaagagtgcatgggtaggcttgatccagaattgataaatctgaaggcaaagctccagggagtaaagcatgaaaatggaagtctacctgagtcagaatcccttcagaagagcatagaagcccggaagaaacagttgttgcctttgtatactcaaattgcggtacggttcgctgaattgcatgacacttcccttagaatggctgctaagggtgtgattaagaaggttgtagactgggaagattctaggtcgttcttctacaagagattacggaggaggatatccgaggatgttcttgcgaaggaaattagaggtgtaagtggcaagcagttttctcaccaatcggcaatcgagctgatccagaaatggtacttggcctctaagggagctgaaacaggaagcactgaatgggatgatgacgatgcttttgttgcctggagggaaaaccctgaaaactaccaggagtatatcaaagaacccagggctcaaagggtatctcagttgctctcagatgttgcagactccagtccagatctagaagccttgccacagggtctttctatgctactagagaagatggatcctgcaaagagggaaattgttgaagactttgaaataaaccttgtaaagtaa

SEQ ID NO: 10 - DNA, 996 bp, Synechococcus sp.
  source 1..996: mol_type = other DNA
atgtcagaggctcaaacgcccctgacagtaccgaagaagtttcttggtgctccaggaggcttcaaccccaccgtcgcactcttcttggcaggttatacctgcgcggcgctctcagttttggggtactggtgctggagttggccccactggctatctttccttctgagtgtcacagccttacatttggtaggcaccgtcattcacgatgcctctcataatgtggctcacgccagtcgcattctgaatgcgattttgggacatggcagtgcactattgctgggctttacttttccggtgtttacgcgggttcacctgcaacatcacgcccacgtcaacgatcccaagaacgatcccgaccacatcgtttccacctttgggccgctgtggttgatcgcaccgcgcttcttctatcacgagatctatttcttccagcgccgcctttggaagaaatttgaattactcgaatggttcctcagtcgcgctgtggtcatcggcatctttgcctgcggcgtcaagtttggcttcctgggcttcctgatgaactactggctggctccagccttggtcgttggcattgccctaggactcttcttcgactatttaccccaccgccccttccaagagcgcaaccgctggcgcaatgcacgggtctatcccggtcaggtgatgaacatcctgatcatgggtcagaactatcacctgatccatcacctctggccatcgatcccctggtatctctaccgaccggcctaccacgctaccaagccgttgttggacctacgccagtcgccgcaaacgctcgggattctctccagcaaaaaagatttctggaactttatctacgacgttttcatcggcatccgcattcaccaatcgcacgaggctgagccgcagagctccgtcgttcctgaaacgaagtcgagtgaatcagccgttctcgcaaaagctccgatgtctgccacagaagactctcgtgagccagccttgacgaagtag

SEQ ID NO: 11 - DNA, 486 bp, Brevundimonas sp.
  source 1..486: mol_type = other DNA; note = Brevundimonas sp. SD212
atggcctggctgacgtggatcgcgctgttcctgaccgcctttttgggcatggaggcgttcgcctggatcatgcaccgctatgtgatgcacggtttcctgtggtcctggcaccgcagccatcatgagccgcacgatcaccccctggagaagaacgacctgttcgccgtggtcttcgccgccccggccatcgtcatggtggccgtgggtctgcacctgtggccctgggccctgccggtcggcctggggatcacggcctatgggatggtctatttcttcttccacgacggcctggtgcaccggcggttcccgacgggcttttccgggcggtccggcttctggacgcggcgcatccaggcgcaccgtctgcatcacgccgtgcgcacgcgcgaaggctgcgtctccttcggctttctgtgggtgcggtcggcgcgggcgctgaaggccgaactggctcagaagcggggctcttccagcagcggcgcctga

SEQ ID NO: 12 - DNA, 735 bp, Brevundimonas sp.
  source 1..735: mol_type = other DNA; note = Brevundimonas sp. SD212
atgaccgccgccgtcgccgagccccgcatcgtcccgcgccagacctggatcggtctgaccctggcgggaatgatcgtggcgggatgggggagcctgcacgtctacggcgtctattttcaccgctggggcacctccagtctggtgatcgtcccggcgatcgtagcggtccagacctggttgtcggtcggccttttcatcgtcgcccatgacgccatgcacggctccctggcgccgggacggccgcggctgaacgccgcagtcggccggctgaccctggggctctatgcgggcttccgcttcgatcggctgaagacggcgcaccacgcccaccacgccgcgcccggcacggccgacgacccggacttttacgccccggcgccccgcgccttccttccctggttcctgaacttctttcgcacctatttcggctggcgcgagatggcggtcctgaccgccctggtcctgatcgccctcttcggcctgggggcgcggccggccaatctcctgaccttctgggccgcgccggccctgctttcagcgcttcagctcttcaccttcggcacctggctgccgcaccgccacaccgaccagccgttcgccgacgcccaccacgcccgcagcagcggctacggccccgttctttccctgctcacctgcttccacttcggccgccaccacgaacaccacctcaccccctggcggccctggtggcgtttgtggcgcggcgagtcttga

SEQ ID NO: 13 - DNA, 23 bp, synthetic construct
  misc_feature 1..23: note = primer to A. plantensis genome outside of homology arm
  source 1..23: mol_type = other DNA
actactaatgtggctttccaggg

SEQ ID NO: 14 - DNA, 22 bp, synthetic construct
  misc_feature 1..22: note = primer to A. plantensis outside of the homology arm
  source 1..22: mol_type = other DNA
gtaaattagcgcgatgggtttg

SEQ ID NO: 15 - DNA, 21 bp, synthetic construct
  misc_feature 1..21: note = primer to homology arm upstream of pilA gene
  source 1..21: mol_type = other DNA
gttggagcaatggcacgagtc

SEQ ID NO: 16 - DNA, 23 bp, synthetic construct
  misc_feature 1..23: note = primer to upstream end of aadA gene
  source 1..23: mol_type = other DNA
atgacgccaactacctctgatag

SEQ ID NO: 17 - DNA, 22 bp, synthetic construct
  misc_feature 1..22: note = primer to downstream end of aadA gene
  source 1..22: mol_type = other DNA
gtatcagcccgtcatacttgaa

SEQ ID NO: 18 - DNA, 22 bp, synthetic construct
  misc_feature 1..22: note = primer to pilA gene
  source 1..22: mol_type = other DNA
gacggtggtttcaccttaatcg

SEQ ID NO: 19 - DNA, 22 bp, synthetic construct
  misc_feature 1..22: note = primer to pilA gene
  source 1..22: mol_type = other DNA
tgctgtagtacctacccagacc

SEQ ID NO: 20 - DNA, 20 bp, synthetic construct
  misc_feature 1..20: note = primer
  source 1..20: mol_type = other DNA
cataccctgctgtgtggcaa

SEQ ID NO: 21 - DNA, 21 bp, synthetic construct
  misc_feature 1..21: note = primer
  source 1..21: mol_type = other DNA
cgggtgatatctttggtggtg

SEQ ID NO: 22 - DNA, 22 bp, synthetic construct
  misc_feature 1..22: note = primer
  source 1..22: mol_type = other DNA
gtacaaatgtacggccagcaac

SEQ ID NO: 23 - DNA, 528 bp, synthetic construct
  misc_feature 1..528: note = codon-optomized Pantoea ananatis beta-carotene 3,3'-hydroxylase (crtZ)
  source 1..528: mol_type = other DNA
atgctgtggatttggaacgcgctgatcgtgtttgtcacagtcatcgggatggaagttattgccgccttggcacacaaatacatcatgcacggctggggctggggatggcatctgtctcaccacgaaccccgcaaaggcgctttcgaagtcaatgatctgtatgctgtcgtgtttgcagccctgagcattttgctcatctacctgggctcgaccggtatgtggccgttgcaatggattggtgccggcatgaccgcctacggtctgctgtatttcatggtgcacgatggcctggtccatcaacggtggccttttcggtatattccccgaaagggctacctgaagcgcctatatatggcccatcgaatgcatcacgcagtccgggggaaggaagggtgtgtgagttttgggtttctatatgctcccccgttgtcgaaactgcaagcaaccctgcgtgagcgtcatggcgctcgcgctggggcggcgcgggacgcgcaagggggcgaagatgaaccggcatcggggaaataa

SEQ ID NO: 24 - DNA, 720 bp, synthetic construct
  misc_feature 1..720: note = codon-optomized Brevundimonas sp. SD212 beta-carotene 4,4'-ketolase (crtW)
  source 1..720: mol_type = other DNA
atgacggctgcggtcgctgagccgcgaatcgtgcctcgtcaaacctggatcgggctcacacttgcagggatgattgtggcaggctgggggagtcttcacgtctatggagtctactttcatcgctgggggacaagtagcctcgttattgtgccagcaatcgtggctgtccagacctggctcagtgtgggcctattcattgtcgcacacgacgctatgcacgggtcgctggcgccgggtcggcctcgcctaaacgcagccgtaggcagactcaccttagggctctatgccggtttccgatttgatcgccttaaaacggcccaccacgcacatcacgccgcccccggtactgccgatgaccctgatttctacgccccagcaccgcgagcgtttttgccttggttcctcaacttctttcgcacttactttggctggcgtgagatggctgttctgacagctttagtcctaatagccttgtttggtttgggtgcccggccggcaaatcttctcacgttctgggcagctccggcgctgttaagtgctctacagctattcacttttggcacgtggctgccgcaccgccacactgatcagccttttgcggacgcccaccatgctcggtcttccggttacggcccggtgctcagcctgcttacctgttttcactttggtcggcaccacgaacaccatctgacaccgtggcgcccctggtggcggctatgg

SEQ ID NO: 25 - DNA, 489 bp, Arthrospira platensis
  source 1..489: mol_type = other DNA
atgaaaacccccctaaccgaagcagtttctatcgctgattcccaaggtcgtttcctaagcagcaccgaaatccaagtagcttttggccgttttcgtcaagccaaagctggtctggaagctgctaaagctttgacctctaaagctgatagtctgatcagtggtgctgcccaagcagtgtacaacaagttcccctacaccacccaaatgcagggacctaactacgcggcagaccaacgcggtaaggacaaatgtgctcgtgacataggctactacctgcggatggtaacttattgcctgattgctggtggaactggccccatggatgagtacctgattgccggtattgatgaaatcaaccggactttcgagctttctccaagctggtacattgaagccctgaaatacatcaaagctaaccacggtttgtctggtgacgctgctgttgaagctaactcctacctcgactacgcgatcaacgccctgagctag

SEQ ID NO: 26 - DNA, 519 bp, Athroisma gracile
  source 1..519: mol_type = other DNA
atgtttgatgccttcaccaaggtggtttctcaagctgatactcgcggcgaaatgctgagtacagctcaaatcgatgctctgagccaaatggttgctgaaagcaacaaacgtttggattctgttaaccgcattaccagcaacgcttccaccattgtttccaacgctgctcgttctttgttcgcagagcaaccccaactgattgctcccggtggaaacgcctacaccagccgtcgtatggctgcttgcttgcgtgacatggaaatcatcctgcgctatgtaacctacgctgtgtttgctggcgatgcaagtgttctcgaagatcgttgcttgaacggtttgcgtgaaacttacctggctttgggaactcccggttcttccgttgctgtcggtgttggcaaaatgaaagaagctgctctggcgatcgttaacgatcccgcaggtatcactcctggtgattgtagcgctttggcttcagaaatcgctggttactttgaccgtgctgctgcagcagtttcctaa

SEQ ID NO: 27 - DNA, 1406 bp, Arthrospira platensis
  source 1..1406: mol_type = genomic DNA
ttgaaggggtgagaaggaaggcgaatgttgatttatgaagtttgattaacatttgtatcaaaatataaaattcttctcataaaccctgtacaatcttttaagatttcggaaagtgttctaggatactgaagaaatgaaccacggggcaattgttaaaagcctttgtcgatggttcgccccggaaggggtcttaggaggtgacaccgatggattgattgtcgtgatcattcatggtgtgtccaatcccaactcaactctaagcaagtcaacaagtaggagataaatccatgtttgatgccttcaccaaggtggtttctcaagctgatactcgcggcgaaatgctgagtacagctcaaatcgatgctctgagccaaatggttgctgaaagcaacaaacgtttggattctgttaaccgcattaccagcaacgcttccaccattgtttccaacgctgctcgttctttgttcgcagagcaaccccaactgattgctcccggtggaaacgcctacaccagccgtcgtatggctgcttgcttgcgtgacatggaaatcatcctgcgctatgtaacctacgctgtgtttgctggcgatgcaagtgttctcgaagatcgttgcttgaacggtttgcgtgaaacttacctggctttgggaactcccggttcttccgttgctgtcggtgttggcaaaatgaaagaagctgctctggcgatcgttaacgatcccgcaggtatcactcctggtgattgtagcgctttggcttcagaaatcgctggttactttgaccgtgctgctgcagcagtttcctaatcaagcagatccatagcatataacaattgaaacagtttagctgaagtctaagtgactggacttctgtttgttacctaattttttgtaaaccaatcgggagataactcgagaatgaaaacccccctaaccgaagcagtttctatcgctgattcccaaggtcgtttcctaagcagcaccgaaatccaagtagcttttggccgttttcgtcaagccaaagctggtctggaagctgctaaagctttgacctctaaagctgatagtctgatcagtggtgctgcccaagcagtgtacaacaagttcccctacaccacccaaatgcagggacctaactacgcggcagaccaacgcggtaaggacaaatgtgctcgtgacataggctactacctgcggatggtaacttattgcctgattgctggtggaactggccccatggatgagtacctgattgccggtattgatgaaatcaaccggactttcgagctttctccaagctggtacattgaagccctgaaatacatcaaagctaaccacggtttgtctggtgacgctgctgttgaagctaactcctacctcgactacgcgatcaacgccctgagctag